(19) United States
(12) Patent Application Publication
(10) Pub. No.: US 2011/0301350 A1
(43) Pub. Date: Dec. 8, 2011

(54) PROCESS FOR PREPARING CYCLOALKYL-SUBSTITUTED PIPERAZINE COMPOUNDS

(75) Inventors: Waldemar Pfrengle, Biberach (DE); Markus Frank, Ulm (DE); Thorsten Pachur, Schwendi (DE); Guenther Huchler, Hochdorf (DE)

(73) Assignee: BOEHRINGER INGELHEIM INTERNATIONAL GMBH, Ingelheim am Rhein (DE)

(21) Appl. No.: 13/058,658

(22) PCT Filed: Feb. 13, 2009
(86) PCT No.: PCT/EP09/01012

§ 371 Date: Aug. 10, 2011

(30) Foreign Application Priority Data
Aug. 12, 2008 (EP) PCT/EP2008/060562

(51) Int. Cl. C07D 295/135; C07D 295/155

(52) U.S. Cl. 544/399; 544/402

(57) ABSTRACT

The present invention relates to a process for preparing compounds of general formula I

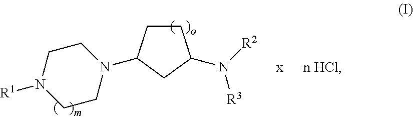

wherein m, n, o, R¹, R² and R³ are defined as mentioned hereinafter, the enantiomers thereof and the diastereomers thereof, which are particularly suitable for preparing compounds of general formula II

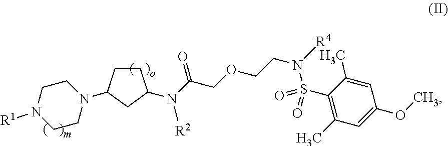

wherein m, o, R¹, R², R³ and R⁴ are defined as mentioned hereinafter. The compounds of general formula II have B1-antagonistic properties.

The present invention relates to a process for preparing compounds of general formula I

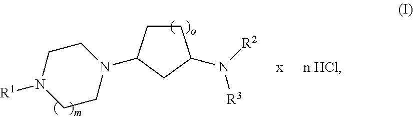

wherein m, n, o, R¹, R² and R³ are defined as mentioned hereinafter, the enantiomers thereof and the diastereomers thereof, which are particularly suitable for preparing compounds of general formula II

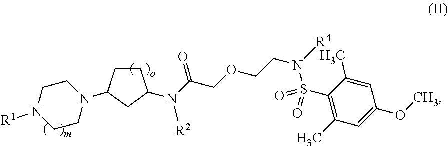

wherein m, o, R¹, R², R³ and R⁴ are defined as mentioned hereinafter. The compounds of general formula II have B1-antagonistic properties.

In a first aspect the present invention relates to a process for preparing compounds of general formula I

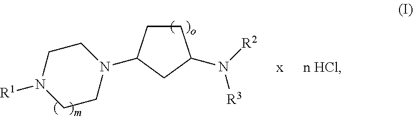

wherein

m denotes one of the numbers 1 or 2,

n denotes one of the numbers 0, 1, 2 or 3,

o denotes one of the numbers 0, 1, 2 or 3,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms, and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms,
   the enantiomers thereof and the diastereomers thereof, comprising the following steps:
(a) addition of a compound of general formula III

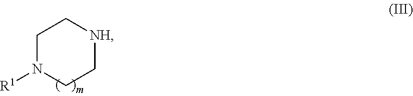

wherein m and R¹ are defined as mentioned hereinbefore, to a compound of general formula IV

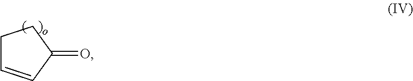

wherein o is defined as mentioned hereinbefore;
(b) reaction of a compound of general formula V obtained in step (a)

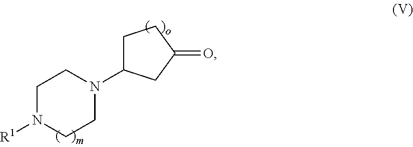

wherein m, o and R¹ are defined as mentioned hereinbefore, with hydroxylamine-hydrochloride;
(c) reduction of an oxime of general formula VI obtained in step (b)

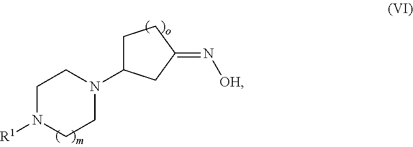

wherein m, o and R¹ are defined as mentioned hereinbefore, in the presence of a catalyst;
(d) optionally isolation of a compound of general formula Ia obtained in step (c)

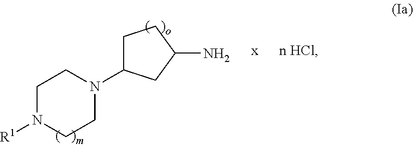

wherein m, n, o and R¹ are defined as mentioned hereinbefore;
(e) coupling an amine of general formula Ia obtained in step (c) or (d)

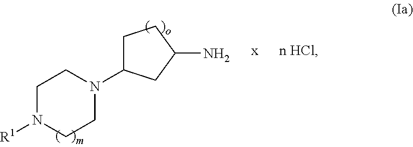

wherein m, n, o and R¹ are defined as mentioned hereinbefore, to a compound of general formula VII

X—R², (VII)
wherein R² is defined as mentioned hereinbefore and X denotes a leaving group, for example a halogen atom, a tosylate, mesylate, triflate or a hydroxysuccinimide-group;
(f) optionally isolating a compound of general formula Ib obtained in step (e)

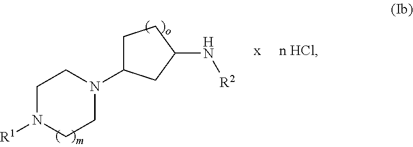

wherein m, n, o, R¹ and R² are defined as mentioned hereinbefore;
(g) optionally again coupling a compound of general formula Ib obtained in step (e) or (f)

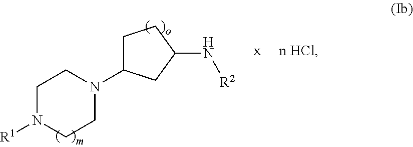

wherein m, n, o, R¹ and R² are defined as mentioned hereinbefore, to a compound of general formula VIII

X—R³, (VIII)
wherein R³ is defined as mentioned hereinbefore and X denotes a leaving group, for example a halogen atom, a tosylate, mesylate, triflate or a hydroxysuccinimide group;
(h) optionally isolating a compound of general formula I obtained in step (g);
(i) optionally stereoselectively separating or concentrating the stereoisomers of a compound of general formula Ia obtained in step (c) or (d) or of a compound of general formula Ib obtained in step (e) or (f) or of a compound of general formula I obtained in step (g) or (h), by co-crystallisation or salt formation with inorganic acids or chiral acids;
(j) optionally isolating one or more stereoisomers of general formula IX obtained in step (i)

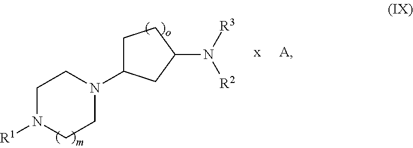

wherein m, n, o, R¹, R² and R³ are defined as mentioned hereinbefore and A denotes one or more chiral acids or one or more corresponding anions of one or more inorganic acids;
(k) reacting a compound of general formula IX obtained in step (i) or (j) with a base;
(l) optionally isolating a stereoisomeric or enantiomerically enriched compound of general formula I

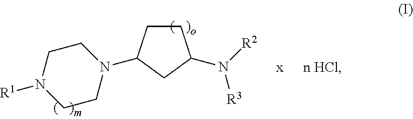

wherein m, n, o, R¹, R² and R³ are defined as mentioned hereinbefore; and
(m) optionally subsequently eliminating an amine protecting group in a compound of general formula I thus obtained wherein m, n and o are defined as mentioned hereinbefore and at least one of the groups R¹, R² or R³ carries an amine protecting group, for example a benzyl, C₁₋₄-alkyl-O—C(O)—, benzyl-O—C(O)—, acetyl, trifluoroacetyl or trichloroacetyl group, thus obtaining a compound of general formula I wherein m, n and o are defined as mentioned hereinbefore and at least one of the groups R¹, R² or R³ denotes a hydrogen atom; and
(n) optionally reducing a compound of general formula I thus obtained wherein m, n, o and R¹ are defined as mentioned hereinbefore and at least one of the groups R² or R³ denotes a C₁₋₄-alkyl-O—C(O)— or benzyl-O—C(O)— group, with a reducing agent, thus obtaining a compound of general formula I wherein m, n, o and R¹ are defined as mentioned hereinbefore and at least one of the groups R² or R³ denotes a methyl group.

The product of each step may be isolated by suitable methods known from the literature, such as e.g. by crystallisation, chromatography or evaporating to dryness.

In the addition in step (a) preferably 1.0 equivalents of a compound of general formula III are reacted with 1.0 to 1.5 equivalents, preferably 1.0 to 1.2 equivalents, of a compound of general formula IV, either without a solvent or in a polar organic solvent. The polar organic solvent used may be methanol, ethanol, propanol, isopropanol, acetone, isopropyl acetate or ethyl acetate or mixtures of these solvents. The solvent is preferably added in an amount of from 0.2 to 0.4 L/mol of compound of general formula III used, preferably in an amount of from 0.25 to 0.35 L/mol of compound of general formula IV used.

In the reaction in step (b) preferably 1.0 equivalents of a compound of general formula V are reacted with 1.0 to 1.5 equivalents, preferably 1.1 to 1.3 equivalents, of hydroxylamine-hydrochloride in a polar organic solvent. The polar organic solvent used may be methanol, ethanol, propanol, isopropanol, acetone, isopropyl acetate or ethyl acetate or mixtures of these solvents. The solvent is preferably added in an amount of from 0.6 to 1.2 L/mol of compound of general formula V used, preferably in an amount of from 0.75 to 1.1 L/mol of compound of general formula V used. The reaction in step (b) may also be carried out in the presence of an inorganic base. The base is preferably added in an amount of from 1.0 to 1.5 equivalents, preferably 1.1 to 1.3 equivalents, based on the amount of compound of general formula V used. Lithium carbonate, sodium carbonate, potassium carbonate or sodium hydrogen carbonate may be used, while potassium carbonate is preferably used according to the invention.

Alternatively to the synthesis described above in steps (a) and (b) a compound of general formula VI may also be prepared by addition of a compound of general formula III

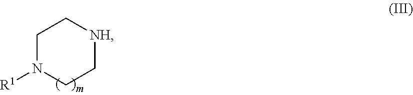

wherein m and R¹ are defined as mentioned hereinbefore, to a compound of general formula IVa

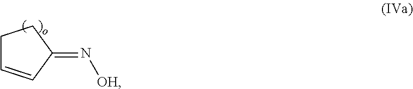

wherein o is defined as mentioned hereinbefore.

In the reduction in step (c) preferably 1.0 equivalents of a compound of general formula VI are reacted in water or an organic solvent in the presence of a reducing agent and optionally in the presence of a base. The organic solvent used may be methanol, ethanol, propanol, butanol, ethyl acetate, toluene, xylene, tetrahydrofuran, methyl-tetrahydrofuran or a mixture of these solvents. The solvent is preferably used in an amount of from 1.5 to 2.5 L/mol of the compound of general formula VI used, preferably from 1.9 to 2.1 L/mol of the compound of general formula VI used. The base is preferably added in an amount of from 0.02 to 0.2 equivalents, preferably 0.07 to 0.15 equivalents, in each case based on the amount of compound of general formula VI used. The base used may be ammonia, triethylamine, diisopropylethylamine or diazabicyclo[5.4.0]undec-7-ene (DBU), while ammonia is preferably used according to the invention.

The reducing agent may be selected from among hydrogen, hydrogen/palladium/charcoal, hydrogen/palladium or hydrogen/Raney nickel, formic acid, formates, complex metal hydrides, sodium/alcohols, zinc/acetic acid, tin/hydrochloric acid, while preferably hydrogen/palladium/charcoal are used. 1 to 3 equivalents, preferably 1.5 to 2.5 equivalents, of the reducing agent may be added, in each case based on the amount of the compound of general formula VI used.

Advantageous conditions for the hydrogenation are temperatures from 20 to 60° C., preferably 25 to 35° C., and an excess hydrogen pressure of at most 5 bar. After the catalyst has been filtered off the hydrogenation product may be concentrated by distilling off the solvent. After working up a compound of general formula Ia is obtained, wherein n denotes the number 0.

The isolation of a compound of general formula Ia described in step (d) may take place in the form of the free amine, while n denotes the number 0.

A compound thus obtained may then be dissolved in a solvent and converted by the addition of a corresponding amount of hydrochloric acid into a compound of general formula Ia, wherein n denotes one of the numbers 1, 2 or 3, preferably the number 3. The solvent used may be methanol, ethanol, propanol, butanol, isopropanol, tert-amylalcohol, isopropyl acetate, tetrahydrofuran, methyltetrahydrofuran, dioxane, ethyl acetate, dichloromethane, methylcyclohexane or toluene.

In the coupling in step (e), preferably 1.0 equivalents of a compound of general formula Ia are reacted with 1.0 to 1.5 equivalents, preferably 1.0 to 1.2 equivalents, of a compound of general formula VII in a solvent and in the presence of a base. The solvent used may be water, methanol, ethanol, propanol, butanol, isopropanol, tert-amylalcohol, acetone, methylcyclohexane, toluene, xylene, tetrahydrofuran, methyltetrahydrofuran, dioxane, ethyl acetate, isopropyl acetate or dichloromethane or mixtures of these solvents. The solvent is preferably added in an amount of from 1.0 to 2.0 L/mol of the compound of general formula Ia used, preferably in an amount of from 1.4 to 1.6 L/mol of the compound of general formula Ia used.

The coupling may also take place in the presence of a base. The base is preferably added in an amount of from 3.0 to 5.0 equivalents, preferably 3.8 to 4.5 equivalents, based on the amount of compound of general formula Ia used. Lithium carbonate, potassium carbonate, sodium carbonate, triethylamine, disopropylethylamine or DBU (diazabicyclo[5.4.0]undec-7-ene) may be used, while potassium carbonate is preferably used according to the invention.

The isolation of a compound of general formula Ib described in step (f) may take place in the form of the free amine, wherein n denotes the number 0.

A compound thus obtained may then be dissolved in a solvent and converted by the addition of a corresponding amount of hydrochloric acid into a compound of general formula Ib, wherein n denotes one of the numbers 1, 2 or 3, preferably the number 3. The solvent used may be water, methanol, ethanol, propanol, butanol, isopropanol, isopropyl acetate, tert-amylalcohol, tetrahydrofuran, methyltetrahydrofuran, dioxane, ethyl acetate, dichloromethane, methylcyclohexane, toluene or a mixture of these solvents.

A compound of general formula Ib obtained in step (f), wherein m, n, o and R¹ are defined as mentioned hereinbefore and R² denotes a benzyloxycarbonyl group, may be converted in the presence of lithium aluminium hydride into a compound of general formula Ib, wherein m, n, o and R¹ are defined as mentioned hereinbefore and R² denotes a methyl group.

In the coupling in step (g), preferably 1.0 equivalents of a compound of general formula Ib are reacted with 1.0 to 1.5 equivalents, preferably 1.0 to 1.2 equivalents, of a compound of general formula VIII in a solvent and in the presence of a base. The solvent used may be water, methanol, ethanol, propanol, butanol, isopropanol acetone, toluene, xylene, tetrahydrofuran, methyltetrahydrofuran, dioxane, ethyl acetate, isopropyl acetate or dichloromethane or mixtures of these solvents. The solvent is preferably added in an amount of from 1.0 to 2.0 L/mol of the compound of general formula Ib used, preferably in an amount of from 1.4 to 1.6 L/mol of the compound of general formula Ib used. The base is preferably added in an amount of from 3.0 to 5.0 equivalents, preferably 3.8 to 4.5 equivalents, based on the amount of compound of general formula Ib used. Lithium carbonate, potassium carbonate, sodium carbonate, sodium hydrogen carbonate, triethylamine, disopropylethylamine or diazabicyclo[5.4.0]undec-7-ene (DBU) may be used, while potassium carbonate is preferably used according to the invention.

The isolation of a compound of general formula I described in step (h) may take place in the form of the free amine, where n denotes the number 0.

A compound of general formula I thus obtained may then be dissolved in a solvent and converted by the addition of a corresponding amount of hydrochloric acid into a compound of general formula I, wherein n denotes one of the numbers 1, 2 or 3, preferably the number 3.

The solvent used may be methanol, ethanol, propanol, butanol, isopropanol, isopropyl acetate, tert-amylalcohol, tetrahydrofuran, methyltetrahydrofuran, dioxane, ethyl acetate, dichloromethane, methylcyclohexane or toluene.

In the isolations described in steps (d), (f) and (h) in the form of the hydrochloride (n=1, 2 or 3), preferably the trihydrochloride (n=3), a corresponding racemic cis-compound is predominantly obtained.

The separation of the enantiomers described in step (i) is carried out in water or an organic solvent or a mixture thereof. The organic solvent may be selected from among methanol, ethanol, propanol, butanol, isopropanol, isopropyl acetate, tert-amylalcohol, tetrahydrofuran, methyl-tetrahydrofuran, dioxane, ethyl acetate, dichloromethane, methylcyclohexane or toluene, and may be used in an amount of from 1.0 to 2.0 L/mol, preferably 1.4 to 1.6 L/mol, per mole of the compound of general formula I or Ia or Ib used. For compounds wherein n denotes one of the numbers 1, 2 or 3, a corresponding amount of a base is added in order to isolate the compound, wherein n denotes the number 0. The base used may be lithium carbonate, potassium carbonate, sodium carbonate or sodium hydrogen carbonate, while potassium carbonate is preferably used according to the invention.

The chiral acid may be used in an amount of from 0.4 to 0.7 mol per mol of the compound of general formula I, Ia or Ib used. The acid may be selected from among chiral amino acids, tartaric acid, derivatives of tartaric acid, chiral sulphonic acids such as for example (S)-(+)-camphorsulphonic acid, camphanic acid, derivatives of camphanic acid, mandelic acid and malic acid. Preferably, according to the invention, (S)-(+)-camphorsulphonic acid is used.

A reaction described in step (k) is preferably carried out in water or an organic solvent or in a mixture of water and an organic solvent. The organic solvent may be selected from among methanol, ethanol, propanol, butanol, isopropanol, isopropyl acetate, tert-amylalcohol, tetrahydrofuran, methyl-tetrahydrofuran, dioxane, ethyl acetate, dichloromethane, methylcyclohexane or toluene. It may be used in an amount of from 4.0 to 7.0 L/mol, preferably 5.0 to 6.5 L/mol of the compound of general formula IX used. The base may be selected from among lithium hydroxide, sodium hydroxide, potassium hydroxide, lithium carbonate, sodium carbonate, potassium carbonate and potassium tert. butoxide. Preferably, according to the invention, sodium hydroxide, potassium carbonate or potassium tert. butoxide is used. These may be added in an amount of from 1.0 to 1.5 equivalents, preferably 1.0 to 1.1 equivalents, based on the amount of the compound of general formula IX used.

The cleaving of an amine protecting group as described in step (m) for compounds of general formula I, wherein m, n, o are defined as mentioned hereinbefore and at least one of the groups R¹, R² and R³ does not denote the hydrogen atom, may be carried out according to methods known from the literature (T. W. Greene, P. G. M. Wuts “Protective Groups in Organic Synthesis”, 3^{rd} Edition, Wiley Interscience).

The reduction described in step (n) is preferably carried out in an organic solvent. The organic solvent may be selected from among tetrahydrofuran, methyltetrahydrofuran, dioxane, methylcyclohexane, xylene and toluene or a mixture of these solvents. It may be used in an amount of from 2.0 to 4.0 l/mol, preferably 2.0 to 3.0 l/mol of the compound of general formula I used.

The reducing agent may be selected from among the complex metal hydrides, lithium aluminium hydride, diisobutylaluminium hydride and sodium borohydride, while lithium aluminium hydride is preferably used according to the invention.

In a second aspect the present invention relates to a method for preparing compounds of general formula I as described in the first aspect hereinbefore, characterised in that

m denotes one of the numbers 1 or 2,

n denotes one of the numbers 0, 1, 2 or 3,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)— and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof and the diastereomers thereof.

In a third aspect the present invention relates to a method for preparing compounds of general formula I as described in the first aspect hereinbefore, characterised in that

m denotes one of the numbers 1 or 2,

n denotes one of the numbers 0, 1 or 2,

o denotes one of the numbers 0, 1 or 2,

R¹ denotes H, C₁₋₄-alkyl, C₃₋₆-cycloalkyl, C₁₋₄-alkyl-O—C(O)—, benzyl-O—C(O)— or benzyl, and

R² denotes
(a) H,
(b) benzyl,
(c) C₁₋₄-alkyl-O—C(O)— or benzyl-O—C(O)—, and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof and the diastereomers thereof.

In a fourth aspect the present invention relates to a method for preparing compounds of general formula I as described in the first aspect hereinbefore, characterised in that

m denotes one of the numbers 1 or 2,

n denotes one of the numbers 1 or 2,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof and the diastereomers thereof.

In a fifth aspect the present invention relates to a method for preparing compounds of general formula I as described in the first aspect hereinbefore, characterised in that

m denotes one of the numbers 1 or 2,

n denotes the number 0,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof and the diastereomers thereof represent.

In a sixth aspect the present invention relates to a method for preparing compounds of general formula I as described in the first aspect hereinbefore, characterised in that

m denotes one of the numbers 1 or 2,

n denotes the number 3,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof and the diastereomers thereof represent.

In a seventh aspect the present invention relates to a method for preparing compounds of general formula I as described in the first aspect hereinbefore, wherein m, n, o and R¹ are defined as in the first, second, third, fourth, fifth or sixth aspect hereinbefore and

R² denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl,
   the enantiomers thereof and the diastereomers thereof.

In an eighth aspect the present invention relates to a method for preparing compounds of general formula I as described in the first aspect hereinbefore, characterised in that

m denotes the number 1,

n denotes one of the numbers 0, 1, 2 or 3,

o denotes one of the numbers 1 or 2,

R¹ denotes H, CH₃, benzyl, tert.butyl-O—C(O)— or benzyl-O—C(O)—,

R² denotes H, CH₃, benzyl, tert.butyl-O—C(O)— or benzyl-O—C(O)—,

R³ denotes H, CH₃, benzyl, tert.butyl-O—C(O)— or benzyl-O—C(O)—,

the enantiomers thereof and the diastereomers thereof.

In a ninth aspect the present invention relates to an alternative method for preparing compounds of general formula I

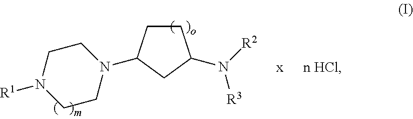

wherein

m denotes one of the numbers 1 or 2,

n denotes one of the numbers 0, 1, 2 or 3,

o denotes the number 2,

R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,

R² denotes
(a) H,
(b) C₁₋₄-alkyl-O—C(O)—, benzyl-O—C(O)— and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—
(e) acetyl, trifluoroacetyl or trichloroacetyl,
   the enantiomers thereof and the diastereomers thereof, comprising the following steps:

(a1) reacting the compound of formula

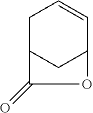

with a compound of general formula III

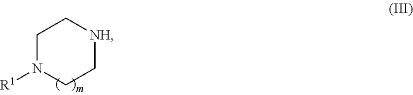

wherein m and R¹ are defined as mentioned hereinbefore, in the presence of a catalyst;

(a2) reducing a compound of general formula X obtained in step (a1)

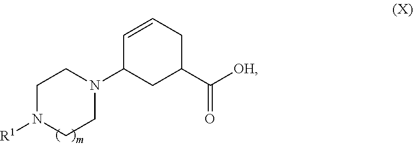

wherein m and R¹ are defined as mentioned hereinbefore;

(a3) reacting a compound of general formula XI obtained in step (a2)

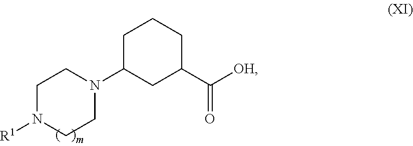

wherein m and R¹ are defined as mentioned hereinbefore, in the presence of a base, with an azide source, for example sodium azide or diphenylphosphoryl azide (DPPA) and catching the resulting intermediate compound of general formula XII

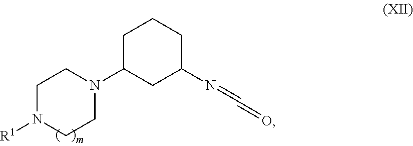

wherein m and R¹ are defined as mentioned hereinbefore, with a compound of general formula XIII

HO—R⁴, (XIII)
wherein R⁴ denotes a hydrogen atom, a C₁₋₄-alkyl or benzyl group; and

(a4) optionally isolating a compound of general formula Ib obtained in step (a3)

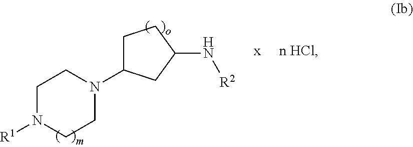

wherein m, n, o, R¹ and R² are defined as mentioned hereinbefore.

The product of each step may be isolated by suitable methods known from the literature, such as e.g. crystallisation, chromatography or evaporating to dryness.

In the reaction in step (a1) preferably 1.0 equivalents of 6-oxa-bicyclo[3.2.1]oct-3-en-7-one are reacted with 1.0 to 1.2 equivalents of a compound of general formula III in water or an organic solvent or mixtures of water and an organic solvent and in the presence of a palladium catalyst PdLₓ (x=0, 1, 2, 3 or 4), a platinum, nickel, copper, cobalt or iridium catalyst and chiral or non-chiral metal ligands, preferably a palladium catalyst PdLₓ (x=0, 1, 2, 3 or 4). The organic solvent used may be methanol, ethanol, propanol, butanol, isopropyl acetate, ethyl acetate, toluene, xylene, tetrahydrofuran, methyltetrahydrofuran or dioxane or a mixture of these solvents. The solvent is preferably added in an amount of from 0.01 to 5.0 mL/mmol, preferably in an amount of from 0.8 to 2.5 mL/mmol, based on the amount of 6-oxa-bicyclo[3.2.1]oct-3-en-7-one used.

The ligand L of the palladium catalyst PdLₓ (x=0, 1, 2, 3 or 4) may be selected from among a halide, chiral or achiral carboxylic acid, olefin, phosphane, amine, or N-heterocyclic carbine ligands or combinations of halide, chiral or achiral carboxylic acid, olefin, phosphane, amine, or N-heterocyclic carbine ligands, the phosphane ligands being e.g. PPh₃, (±)-2,2′-bis(diphenylphosphino)-1,1′-binaphthaline, (R)-(+)-2,2′-bis(diphenylphosphino)-1,1′-binaphthaline, (S)-(−)-2,2′-bis(diphenylphosphino)-1,1′-binaphthaline, (1R,2R)-(+)-1,2-diaminocyclohexane-N,N′-bis(2-diphenylphosphinobenzoyl), (1S,2S)-(−)-1,2-diaminocyclohexane-N,N′-bis(2-diphenylphosphinobenzoyl) or acetate, while dibenzylideneacetone is preferably used according to the invention.

Depending on the nature or choice of the catalyst, it is possible in this reaction step to achieve a concentration of the racemic cis or trans isomers of the compound of general formula X obtained in each case.

The palladium catalyst is preferably added in an amount of from 0.001 to 0.1 equivalents, based on the amount of 6-oxa-bicyclo[3.2.1]oct-3-en-7-one used.

In the reduction in step (a2), preferably 1.0 equivalents of a compound of general formula X are reacted in an organic solvent in the presence of a reducing agent. The organic solvent used may be methanol, ethanol, propanol, ethyl acetate, toluene, xylene, tetrahydrofuran or methyltetrahydrofuran as well as water or a mixture of these solvents. The solvent is preferably used in an amount of from 3 to 6 mL/mmol of the compound of general formula X used, preferably from 4 to 5 mL/mmol of the compound of general formula X used.

The reducing agent may be selected from among hydrogen, hydrogen/charcoal/palladium, hydrogen/palladium, hydrogen/Raney nickel, formic acid and formates, for example alkali metal formates or ammonium formate, while preferably hydrogen/charcoal/palladium is used. 1 to 5 equivalents, preferably 1 to 2 equivalents, of the reducing agent may be added, in each case based on the amount of the compound of general formula X used. Advantageous conditions for the hydrogenation are temperatures of from 20 to 60° C., preferably 25 to 35° C., and an excess hydrogen pressure of at most 5 bar.

After the catalyst has been filtered off the hydrogenation product may be concentrated by distilling off the solvent.

In the reaction in step (a3), preferably 1.0 equivalents of a compound of general formula XI are reacted with 1.0 to 1.5 equivalents, preferably 1.0 to 1.2 equivalents, of a compound of general formula XIII in a solvent and in the presence of a base. The solvent used may be toluene, xylene, tetrahydrofuran, methyltetrahydrofuran, dioxane, ethyl acetate, isopropyl acetate or dichloromethane or mixtures of these solvents. The solvent is preferably added in an amount of from 2.0 to 5.0 mL/mmol of the compound of general formula XI used, preferably in an amount of from 3.0 to 4.0 mL/mmol of the compound of general formula XI used. The base is preferably added in an amount of from 1.0 to 3.0 equivalents, preferably 1.0 to 2.0 equivalents, based on the amount of compound of general formula XI used. Lithium carbonate, potassium carbonate, sodium carbonate, triethylamine, diisopropylethylamine or DBU (diazabicyclo[5.4.0]undec-7-ene) may be used, while triethylamine or diisopropylethylamine is preferably used according to the invention.

The isolation of a compound of general formula I described in step (a4) may take place in the form of the free amine, while n denotes the number 0.

A compound thus obtained may then be dissolved in a solvent and converted by the addition of a corresponding amount of hydrochloric acid into a compound of general formula I, wherein n denotes one of the numbers 1, 2 or 3.

The solvent used may be water, methanol, ethanol, propanol, isopropanol, butanol, ethyl acetate, isopropyl acetate, tetrahydrofuran, methyltetrahydrofuran, dioxane, toluene, acetonitrile, dichloromethane or methylcyclohexane.

In a tenth aspect the present invention relates to a method of preparing compounds of general formula I as described hereinbefore in the ninth aspect, characterised in that

m denotes the number 1,

n denotes one of the numbers 0, 1, 2 or 3,

o denotes the number 2,

R¹ denotes H, CH₃, benzyl, tert.butyl-O—C(O)— or benzyl-O—C(O)—,

R² denotes H, tert.butyl-O—C(O)— or benzyl-O—C(O)—,

R³ denotes H, CH₃, benzyl, tert.butyl-O—C(O)— or benzyl-O—C(O)—,

the enantiomers thereof and the diastereomers thereof.

In an eleventh aspect the present invention relates to an alternative method of preparing compounds of general formula I

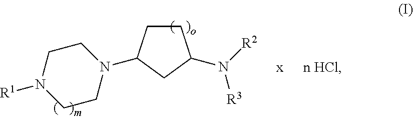

wherein

m denotes one of the numbers 1 or 2,

n denotes one of the numbers 0, 1, 2 or 3,

o denotes the number 2,

R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,

R² denotes
(a) H,
(b) C₁₋₄-alkyl-O—C(O)—, benzyl-O—C(O)— and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—
(e) acetyl, trifluoroacetyl or trichloroacetyl,
   the enantiomers thereof and the diastereomers thereof, comprising the following steps:

(b1) reacting the compound of formula

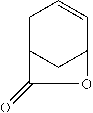

with a compound of general formula III

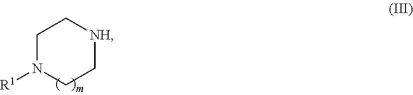

wherein m and R¹ are defined as mentioned hereinbefore, in the presence of a catalyst;

(b2) reacting a compound of general formula X obtained in step (b1)

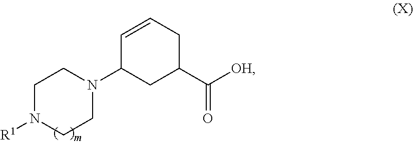

wherein m and R¹ are defined as mentioned hereinbefore, in the presence of a base with an azide source, for example sodium azide or diphenylphosphoryl-azide (DPPA), and catching the resulting intermediate compound of general formula XIV

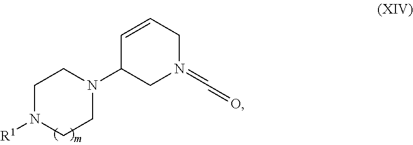

wherein m and R¹ are defined as mentioned hereinbefore, with a compound of general formula XIII

HO—R⁴, (XIII)
wherein R⁴ denotes a hydrogen atom, a C₁₋₄-alkyl or benzyl group;

(b3) optionally isolating a compound of general formula XV obtained in step (b2)

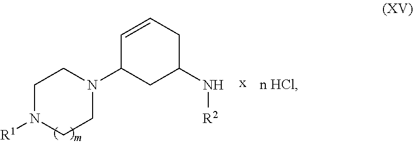

wherein m, n, R¹ and R² are defined as mentioned hereinbefore;

(b4) reducing a compound of general formula XV obtained in step (b2) or (b3)

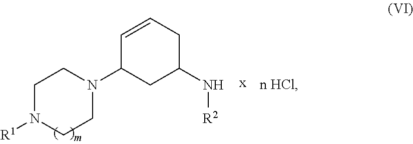

wherein m and R¹ are defined as mentioned hereinbefore; and

(b5) optionally isolating a compound of general formula I obtained in step (b4)

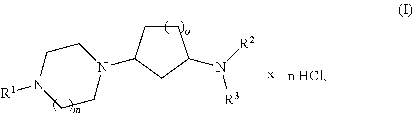

wherein m, n, R¹ and R² are defined as mentioned hereinbefore.

The product of each step may be isolated by suitable methods known from the literature, such as e.g. crystallisation, chromatography or evaporating to dryness.

In the reaction in step (b1) preferably 1.0 equivalents of 6-oxa-bicyclo[3.2.1]oct-3-en-7-one are reacted with 1.0 to 1.2 equivalents of a compound of general formula III in water or an organic solvent or mixtures of water and an organic solvent and in the presence of a palladium catalyst PdLₓ (x=0, 1, 2, 3 or 4), a platinum, nickel, copper, cobalt or iridium catalyst and chiral or non-chiral metal ligands, preferably a palladium catalyst PdLₓ (x=0, 1, 2, 3 or 4). The organic solvent used may be methanol, ethanol, propanol, butanol, isopropyl acetate, ethyl acetate, toluene, xylene, tetrahydrofuran, methyltetrahydrofuran or dioxane or a mixture of these solvents. The solvent is preferably added in an amount of from 0.01 to 5.0 mL/mmol, preferably in an amount of from 0.8 to 2.5 mL/mmol, based on the amount of 6-oxa-bicyclo[3.2.1]oct-3-en-7-one used.

The ligand L of the palladium catalyst PdLₓ (x=0, 1, 2, 3 or 4) may be selected from among a halide, chiral or non-chiral carboxylic acid, olefin, phosphane, amine, or N-heterocyclic carbene ligands or combinations of halide, chiral or non-chiral carboxylic acid, olefin, phosphane, amine, or N-heterocyclic carbene ligands, the phosphane ligands being e.g. PPh₃, (±)-2,2′-bis(diphenylphosphino)-1,1′-binaphthalene, (R)-(+)-2,2′-bis(diphenylphosphino)-1,1′-binaphthalene, (S)-(−)-2,2′-bis(diphenylphosphino)-1,1′-binaphthalene, (1R,2R)-(+)-1,2-diaminocyclohexane-N,N′-bis(2-diphenylphosphinobenzoyl), (1S,2S)-(−)-1,2-diaminocyclohexane-N,N′-bis(2-diphenylphosphinobenzoyl) or acetate, while dibenzylideneacetone is preferably used according to the invention.

Depending on the nature or choice of the catalyst, it is possible in this reaction step to achieve a concentration of the racemic cis or trans isomers of the compound of general formula X obtained in each case.

The palladium catalyst is preferably added in an amount of from 0.001 to 0.1 equivalents, based on the amount of 6-oxa-bicyclo[3.2.1]oct-3-en-7-one used.

In the reaction in step (b2) preferably 1.0 equivalents of a compound of general formula X are reacted with 1.0 to 1.5 equivalents, preferably 1.0 to 1.2 equivalents, of a compound of general formula XIII in a solvent and in the presence of a base. The solvent used may be toluene, xylene, tetrahydrofuran, methyltetrahydrofuran, dioxane, ethyl acetate, isopropyl acetate or dichloromethane or mixtures of these solvents. The solvent is preferably added in an amount of from 2.0 to 5.0 mL/mmol of the compound of general formula X used, preferably in an amount of from 3.0 to 4.0 mL/mmol of the compound of general formula X used. The base is preferably added in an amount of from 1.0 to 3.0 equivalents, preferably 1.0 to 2.0 equivalents, based on the amount of compound of general formula X used. Lithium carbonate, potassium carbonate, sodium carbonate, triethylamine, diisopropylethylamine or DBU (diazabicyclo[5.4.0]undec-7-ene) may be used, while triethylamine or diisopropylethylamine is preferably used according to the invention.

In the reduction in step (b4) preferably 1.0 equivalents of a compound of general formula XV are reacted in an organic solvent in the presence of a reducing agent. The organic solvent may be methanol, ethanol, propanol, ethyl acetate, toluene, xylene, tetrahydrofuran or methyltetrahydrofuran as well as water or a mixture of these solvents.

The solvent is preferably used in an amount of from 3 to 6 mL/mmol of the compound of general formula XV used, preferably from 4 to 5 mL/mmol of the compound of general formula XV used.

The reducing agent may be selected from among hydrogen, hydrogen/charcoal/palladium, hydrogen/palladium, hydrogen/Raney nickel, formic acid and formates, for example alkali metal formates or ammonium formate, while preferably hydrogen/charcoal/palladium is used. 1 to 5 equivalents, preferably 1 to 2 equivalents, of the reducing agent may be added, in each case based on the amount of compound of general formula XV used.

Advantageous conditions for the hydrogenation are temperatures of 20 to 60° C., preferably 25 to 35° C., and an excess hydrogen pressure of at most 5 bar.

After the catalyst has been filtered off the hydrogenation product may be concentrated by distilling off the solvent.

The isolation of a compound of general formula I described in step (b5) may take place in the form of the free amine, while n denotes the number 0.

A compound thus obtained may then be dissolved in a solvent and converted by the addition of a corresponding amount of hydrochloric acid into a compound of general formula I, wherein n denotes one of the numbers 1 or 2.

The solvent used may be water, methanol, ethanol, propanol, isopropanol, butanol, ethyl acetate, isopropyl acetate, tetrahydrofuran, methyltetrahydrofuran, dioxane, toluene, acetonitrile, dichloromethane or methylcyclohexane.

In a twelfth aspect the present invention relates to a method of preparing compounds of general formula I as described hereinbefore in the ninth aspect, characterised in that

m denotes the number 1,

n denotes one of the numbers 0, 1, 2 or 3,

o denotes the number 2,

R¹ denotes H, CH₃, benzyl, tert.butyl-O—C(O)— or benzyl-O—C(O)—,

R² denotes H, tert.butyl-O—C(O)— or benzyl-O—C(O)—,

R³ denotes H, CH₃, benzyl, tert.butyl-O—C(O)— or benzyl-O—C(O)—,

the enantiomers thereof and the diastereomers thereof.

In a thirteenth aspect the present invention relates to the compounds of general formula I wherein

m denotes one of the numbers 1 or 2,

n denotes one of the numbers 0, 1, 2 or 3,

o denotes one of the numbers 0, 1, 2 or 3,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms, and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms,
   the enantiomers thereof, the diastereomers thereof and the salts thereof and co-crystals with chiral acids, preferably the camphorsulphonates thereof.

The compounds of general formula I are valuable starting materials for synthesizing the compounds of general formula II

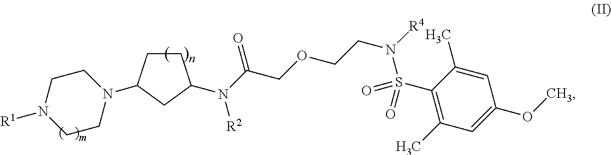

wherein m, n, R¹ and R² are defined as mentioned hereinbefore, R⁴ denotes a hydrogen atom or a methyl group, which have B1-antagonistic properties.

The following are examples of other preferred compounds of general formula I:

| No. | Structure |
|---|---|
| (1) | 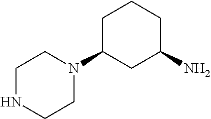 |
| (2) | 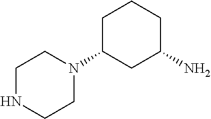 |
| (3) | 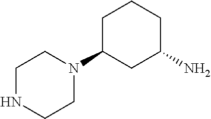 |
| (4) | 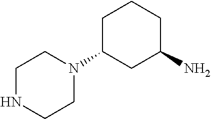 |
| (5) | 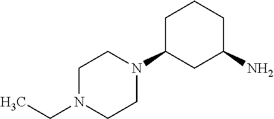 |
| (6) | 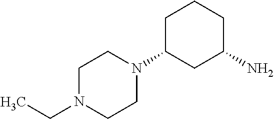 |
| (7) | 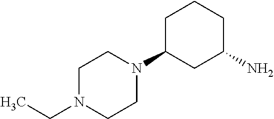 |
| (8) | 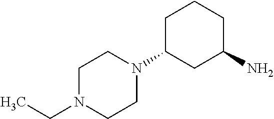 |
| (9) | 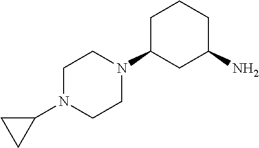 |
| (10) | 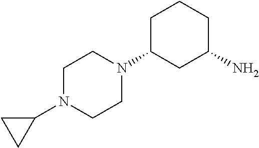 |
| (11) | 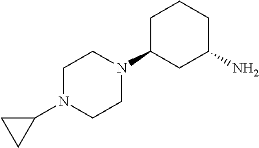 |
| (12) | 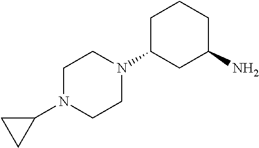 |
| (13) | 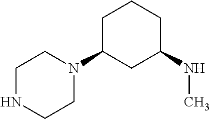 |
| (14) | 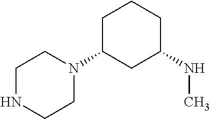 |
| (15) | 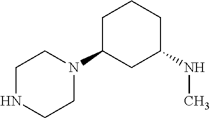 |
| (16) | 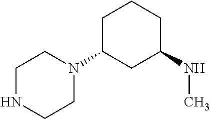 |
| (17) | 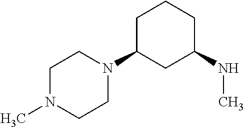 |
| (18) | 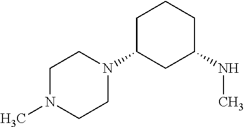 |
| (19) | 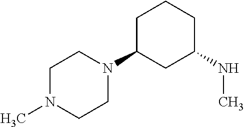 |
| (20) | 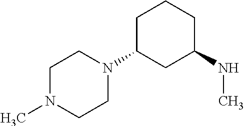 |
| (21) | 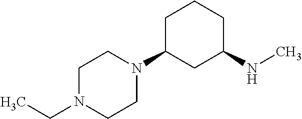 |
| (22) | 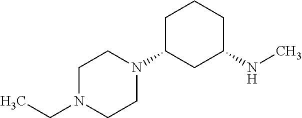 |
| (23) | 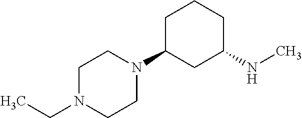 |
| (24) | 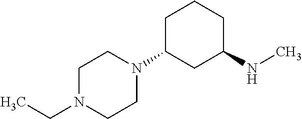 |
| (25) | 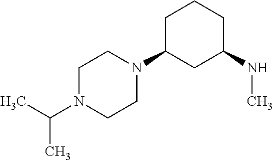 |
| (26) | 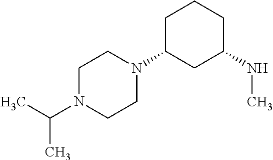 |
| (27) | 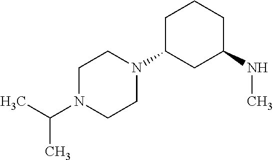 |
| (28) | 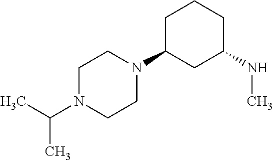 |
| (29) | 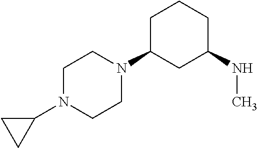 |
| (30) | 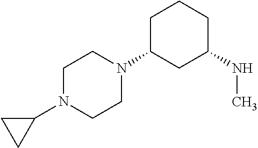 |
| (31) | 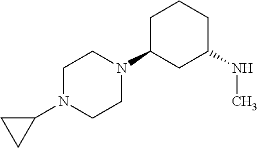 |
| (32) | 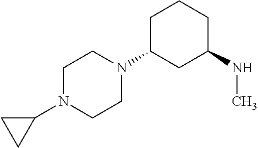 |
| (33) | 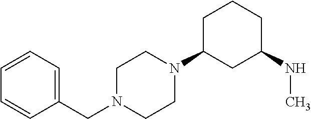 |
| (34) | 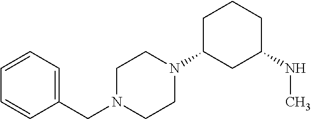 |
| (35) | 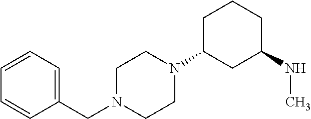 |
| (36) | 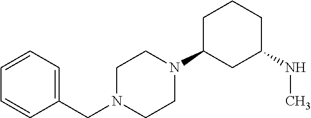 |
| (37) | 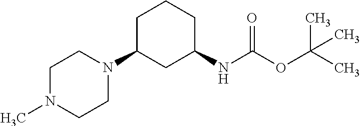 |
| (38) | 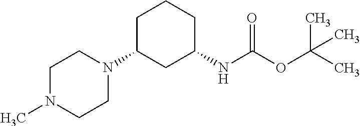 |
| (39) | 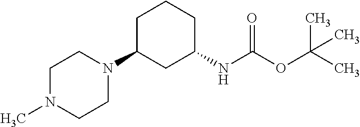 |
| (40) | 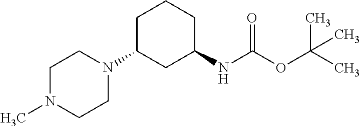 |
| (41) | 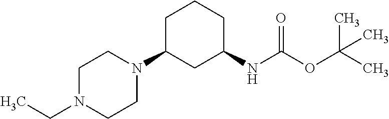 |
| (42) | 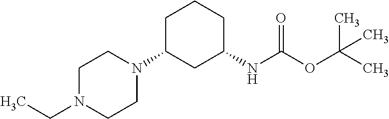 |
| (43) | 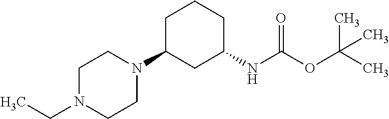 |
| (44) | 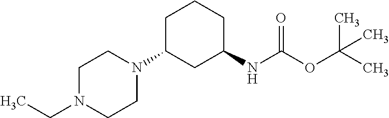 |
| (45) | 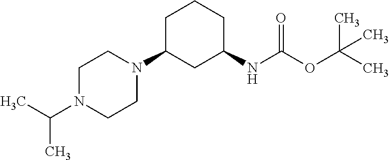 |
| (46) | 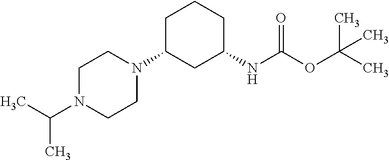 |
| (47) | 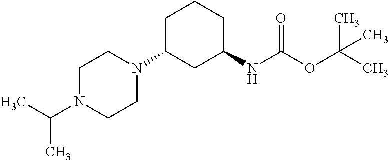 |
| (48) | 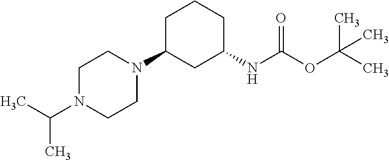 |
| (49) | 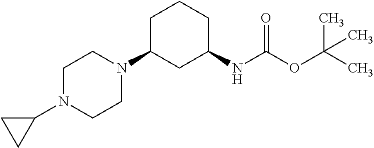 |
| (50) | 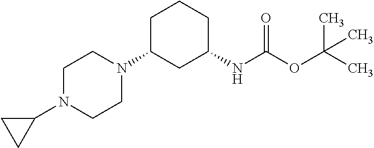 |
| (51) | 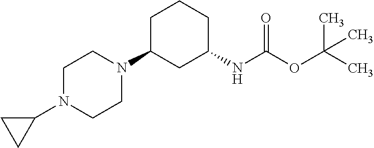 |
| (52) | 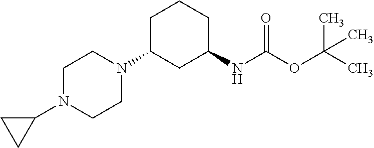 |
| (53) | 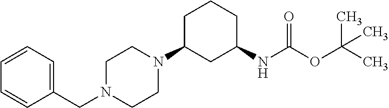 |
| (54) | 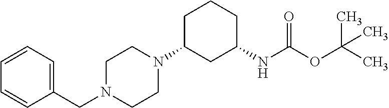 |
| (55) | 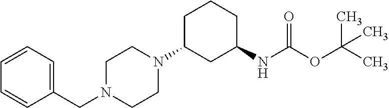 |
| (56) | 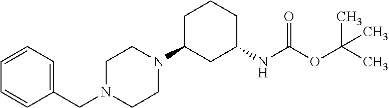 |
| (57) | 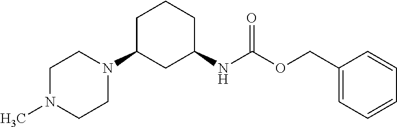 |
| (58) | 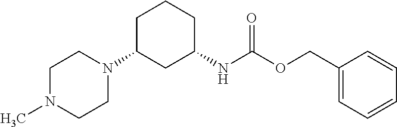 |
| (59) | 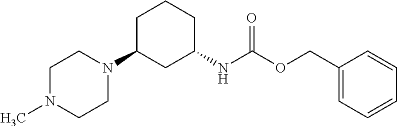 |
| (60) | 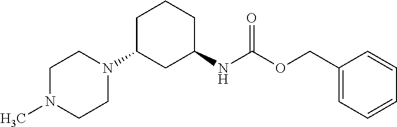 |
| (61) | 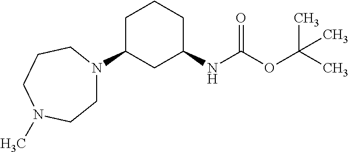 |
| (62) | 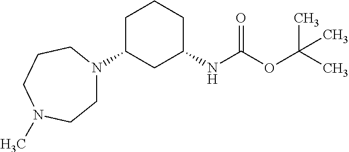 |
| (63) | 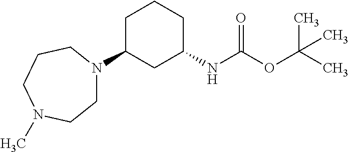 |
| (64) | 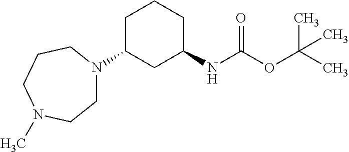 |
| (65) | 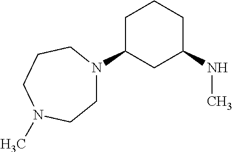 |
| (66) | 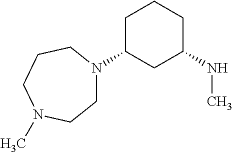 |
| (67) | 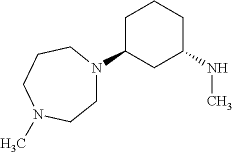 |
| (68) | 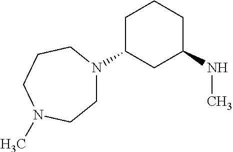 |
| (69) | 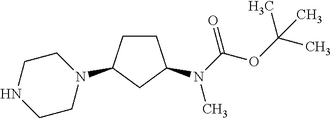 |
| (70) | 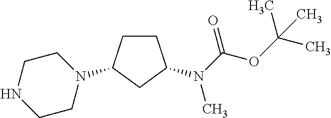 |
| (71) | 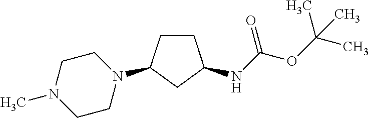 |
| (72) | 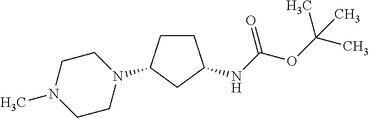 |
| (73) | 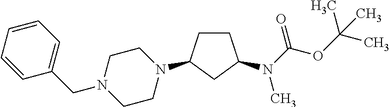 |
| (74) | 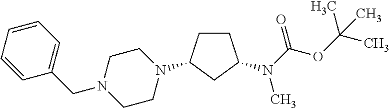 |
| (75) | 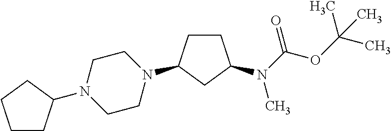 |
| (76) | 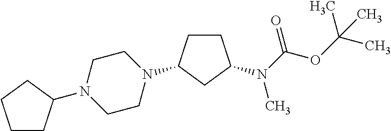 |
| (77) | 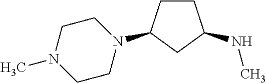 |
| (78) | 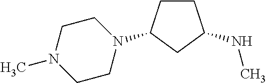 |
| (79) | 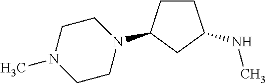 |
| (80) | 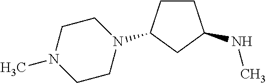 |
| (81) | 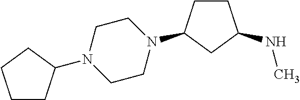 |
| (82) | 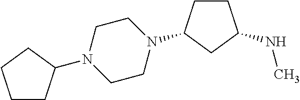 |
| (83) | 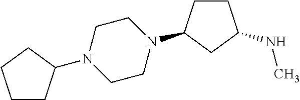 |
| (84) | 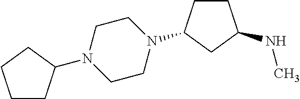 |
| (85) | 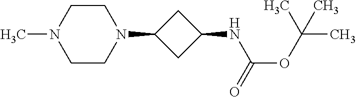 |
| (86) | 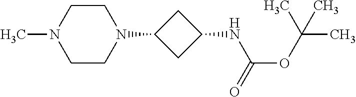 |
| (87) | 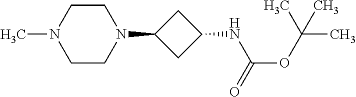 |
| (88) | 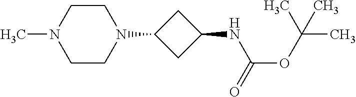 |
| (89) | 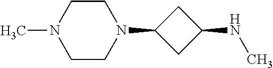 |
| (90) | 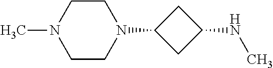 |
| (91) | 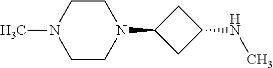 |
| (92) | 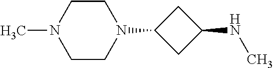 |

the enantiomers thereof, the diastereomers thereof and the salts thereof and co-crystals with chiral acids, preferably the camphorsulphonates thereof.

In a fourteenth aspect the present invention relates to the compounds of general formula I wherein

m denotes one of the numbers 1 or 2,

n denotes one of the numbers 0, 1, 2 or 3,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)— and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof, the diastereomers thereof and the salts thereof and co-crystals with chiral acids, preferably the camphorsulphonates thereof.

In a fifteenth aspect the present invention relates to the compounds of general formula I wherein

m denotes one of the numbers 1 or 2,

n denotes one of the numbers 0, 1 or 2,

o denotes one of the numbers 0, 1 or 2,

R¹ denotes H, C₁₋₄-alkyl, C₃₋₆-cycloalkyl, C₁₋₄-alkyl-O—C(O)—, benzyl-O—C(O)— or benzyl, and

R² denotes
(a) H,
(b) benzyl,
(c) C₁₋₄-alkyl-O—C(O)— or benzyl-O—C(O)—, and

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof, the diastereomers thereof and the salts thereof and co-crystals with chiral acids, preferably the camphorsulphonates thereof.

In a sixteenth aspect the present invention relates to the compounds of general formula I wherein

m denotes one of the numbers 1 or 2,

n denotes one of the numbers 1 or 2,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof, the diastereomers thereof and the salts thereof and co-crystals with chiral acids, preferably the camphorsulphonates thereof.

In a seventeenth aspect the present invention relates to the compounds of general formula I wherein

m denotes one of the numbers 1 or 2,

n denotes the number 0,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof, the diastereomers thereof and the salts thereof and co-crystals with chiral acids, preferably the camphorsulphonates thereof.

In an eighteenth aspect the present invention relates to the compounds of general formula I wherein

m denotes one of the numbers 1 or 2,

n denotes the number 3,

o denotes one of the numbers 0, 1 or 2,

R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—,

R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
   the enantiomers thereof, the diastereomers thereof and the salts thereof and co-crystals with chiral acids, preferably the camphorsulphonates thereof.

In a nineteenth aspect the present invention relates to the compounds of general formula I wherein m, n, o and R¹ are as hereinbefore defined in the thirteenth, fourteenth, fifteenth, sixteenth, seventeenth or eighteenth aspect and

R² denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl and

R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl,
   the enantiomers thereof, the diastereomers thereof and the salts thereof and co-crystals with chiral acids, preferably the camphorsulphonates thereof.

In a twentieth aspect the present invention relates to the use of the previously mentioned compounds of general formula I wherein m, n, o, R¹, R² and R³ are defined as mentioned hereinbefore, as intermediate products for preparing compounds of general formula II, wherein m, o, R¹ and R² are defined as mentioned hereinbefore and R⁴ denotes a hydrogen atom or a C₁₋₃-alkyl group.

In a twenty-first aspect the present invention relates to the compounds of general formula X

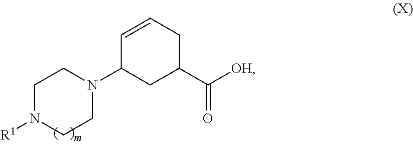

wherein

m denotes one of the numbers 1 or 2 and

R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trichloroacetyl or trifluoroacetyl,
   the enantiomers thereof and the diastereomers thereof as well as the salts and co-crystals thereof with chiral or inorganic acids.

The following are examples of more preferred compounds of general formula X:

| No. | Structure |
|---|---|
| (1) | 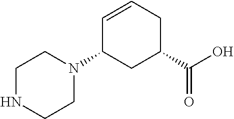 |
| (2) | 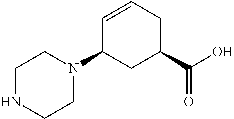 |
| (3) | 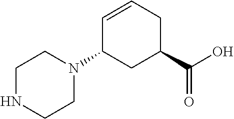 |
| (4) | 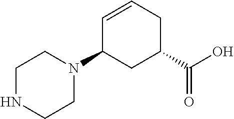 |
| (5) | 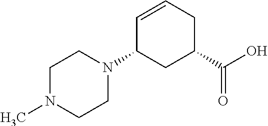 |
| (6) | 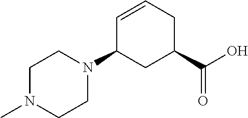 |
| (7) | 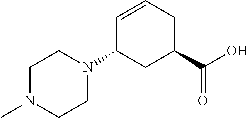 |
| (8) | 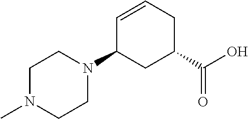 |
| (9) | 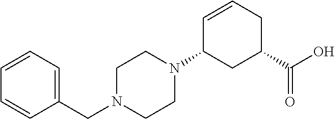 |
| (10) | 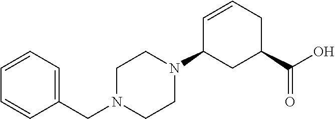 |
| (11) | 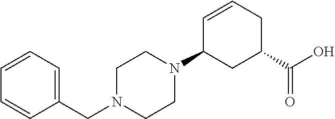 |
| (12) | 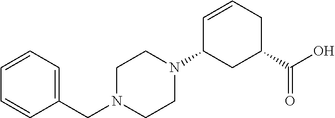 |

the enantiomers thereof and the diastereomers thereof as well as the salts and co-crystals thereof with chiral or inorganic acids.

In a twenty-second aspect the present invention relates to the use of the previously mentioned compounds of general formula X, wherein m and R¹ are defined as mentioned hereinbefore, as intermediate products for preparing compounds of general formula II, wherein m, o, R¹ and R² are defined as mentioned hereinbefore and R⁴ denotes a hydrogen atom or a C₁₋₃-alkyl group.

In a twenty-third aspect the present invention relates to the compounds of general formula XI

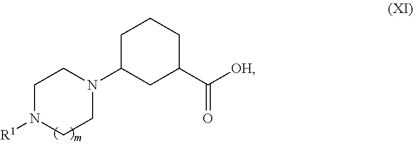

wherein

m denotes one of the numbers 1 or 2 and

R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trichloroacetyl or trifluoroacetyl,
   the enantiomers thereof and the diastereomers thereof as well as the salts and co-crystals thereof with chiral or inorganic acids.

The following are mentioned as examples of more preferred compounds of general formula XI:

| No. | Structure |
|---|---|
| (1) | 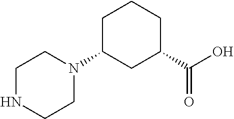 |
| (2) | 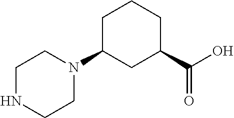 |
| (3) | 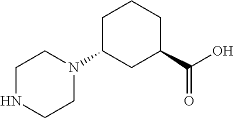 |
| (4) | 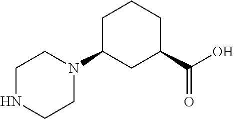 |
| (5) | 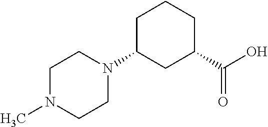 |
| (6) | 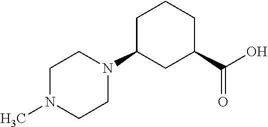 |
| (7) | 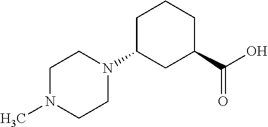 |
| (8) | 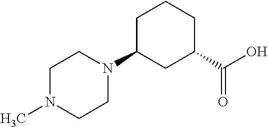 |
| (9) | 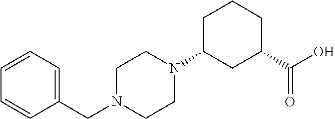 |
| (10) | 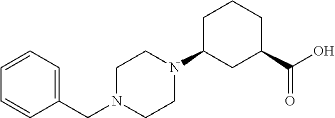 |
| (11) | 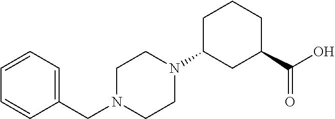 |
| (12) | 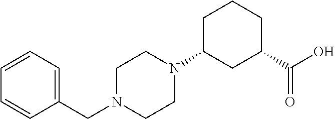 |

the enantiomers thereof and the diastereomers thereof as well as the salts and co-crystals thereof with chiral or inorganic acids.

In a twenty-fourth aspect the present invention relates to the use of the previously mentioned compounds of general formula XI, wherein m and R¹ are defined as mentioned hereinbefore, as intermediate products for preparing compounds of general formula II, wherein m, o, R¹ and R² are defined as mentioned hereinbefore and R⁴ denotes a hydrogen atom or a C₁₋₃-alkyl group.

### Terms and Definitions Used

Also included in the subject-matter of this invention are the compounds according to the invention, including their salts, wherein one or more hydrogen atoms, for example one, two, three, four or five hydrogen atoms, are replaced by deuterium.

Also included in the subject-matter of this invention are the compounds according to the invention, including their salts, wherein one or more ¹³C carbon atoms are replaced by ¹⁴C.

By the term “C₁₋₃-alkyl” (including those that are part of other groups) are meant branched and unbranched alkyl groups with 1 to 3 carbon atoms and by the term “C₁₋₄-alkyl” are meant branched and unbranched alkyl groups with 1 to 4 carbon atoms. Examples include: methyl, ethyl, n-propyl, iso-propyl, n-butyl, iso-butyl or tert-butyl. The abbreviations Me, Et, n-Pr, i-Pr, n-Bu, i-Bu, tert-Bu, etc. are optionally also used for the above-mentioned groups.

By the term “C₃₋₆-cycloalkyl” (including those that are part of other groups) are meant cycloalkyl groups with 3 to 6 carbon atoms. Examples include: cyclopropyl, cyclobutyl, cyclopentyl or cyclohexyl.

By the term “amine protecting group” is meant, for the purposes of the invention, a benzyl, C₁₋₄-alkyl-O—C(O)—, benzyl-O—C(O)—, acetyl, trifluoroacetyl or a trichloroacetyl group.

The compounds of general formula I may have basic groups such as e.g. amino functions. They may therefore be present as internal salts, as salts with pharmaceutically acceptable inorganic acids such as for example hydrobromic acid, phosphoric acid, nitric acid, hydrochloric acid, sulphuric acid, methanesulphonic acid, ethanesulphonic acid, benzenesulphonic acid, p-toluenesulphonic acid or organic acids, such as for example malic acid, succinic acid, acetic acid, fumaric acid, maleic acid, mandelic acid, lactic acid, tartaric acid or citric acid.

Preferably, the compounds of general formula I may be present as salts or co-crystals with chiral organic acids. Suitable chiral acids include in particular chiral amino acids, tartaric acid, derivatives of tartaric acid, chiral sulphonic acids such as for example (S)-(+)-camphorsulphonic acid, camphanic acid, derivatives of camphanic acid, mandelic acid or malic acid, while (S)-(+)-camphorsulphonic acid is of exceptional importance.

The invention relates to the respective compounds optionally in the form of the individual optical isomers, enantiomers or diastereomers, mixtures of the individual enantiomers or racemates, in the form of the tautomers as well as in the form of the free bases or the corresponding acid addition salts.

### Experimental Section

### Preparation of the End Compound

### EXAMPLE 1.1

### 3-(4-methyl-piperazin-1-yl)-cyclohexanone-oxime (D)

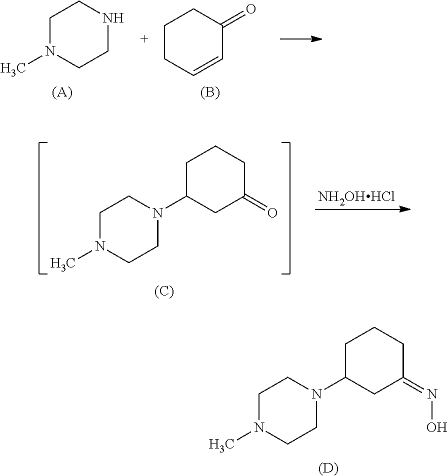

10.00 kg (104.03 mol) 2-cyclohexenone (B) and 10.42 kg (104.03 mol) N-methylpiperazine (A) were stirred for approx. 1 hour at ambient temperature in 30.0 L ethanol. Then the mixture was diluted with 60.0 L ethanol and cooled to 0° C.±5° C. After batchwise addition of 16.17 kg (117.03 mol) potassium carbonate and 8.13 kg (117.03 mol) hydroxylamine hydrochloride the reaction mixture was stirred for approx. 30 minutes at 0° C.±5° C. and then for a further 30 minutes approx. at ambient temperature. The suspension was filtered and diluted with 40.0 L ethanol, before 110 L solvent were distilled off. The residue was diluted with 60.0 L tetrahydrofuran, filtered and diluted with another 20.0 L of tetrahydrofuran, before 40.0 L solvent were distilled off again at normal pressure. 142.0 L n-heptane were added to the residue and the reaction mixture was slowly cooled to ambient temperature. At 40° C.±5° C. the mixture was inoculated. After the suspension had been stirred for approx. 12 to 15 hours at ambient temperature, a further 102.0 L n-heptane were added, the mixture was stirred for approx. 1 hour at ambient temperature and for approx. 1 hour at 0° C.±5° C. The product (D) was filtered off, washed twice with 36.0 L of n-heptane and dried at 50° C.±5° C.

Yield: 18.25 kg (83% of theory)

melting point: 108-110° C.

### EXAMPLE 1.2

### rac-cis-3-(4-methyl-piperazin-1-yl)-cyclohexylamine trihydrochloride (E)

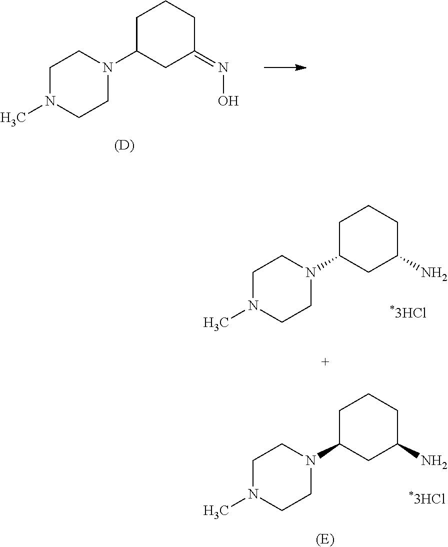

A mixture of 10.00 kg (47.32 mol) 3-(4-methyl-piperazin-1-yl)-cyclohexanone-oxime (D), 45.0 L toluene, 45.0 L ethanol and 0.8 L ethanolic ammonia was hydrogenated with 1.40 kg of Raney nickel and hydrogen at approx. 5 bar and at 30° C.±5° C. until all the hydrogen had been absorbed. Then the mixture was filtered and diluted with 20.0 L ethanol and methanol, before the solvent was completely distilled off in vacuo. After the addition of 20.0 L methanol the solvent was distilled off again in vacuo. The residue was diluted with 50.0 L methanol, heated to 50° C.±5° C. and combined with 13.27 kg (141.96 mol, 10 molar) ethanolic hydrochloric acid. After inoculation and approx. 30 minutes stirring the suspension was cooled to ambient temperature, the product was separated off from the solvent and washed with 10.0 L cold methanol. After recrystallisation from 50.0 L methanol the racemic cis-product (E) was dried at 45° C.±5° C.

Yield: 6.24 kg (43% of theory)

melting point: 254-256° C. (decomposition)

### EXAMPLE 1.3

### [(1S,3R)-3-(4-methyl-piperazin-1-yl)-cyclohexyl]carbamidic acid benzylester-[(1S,4R)-7,7-dimethyl-2-oxo-bicyclo[2.2.1]hept-1-yl]-methanesulphonate (G)

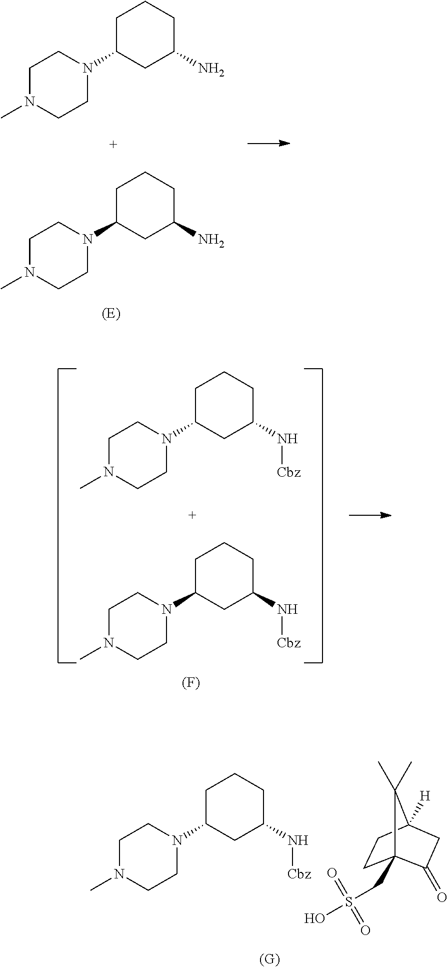

19.15 kg (138.59 mol) potassium carbonate were dissolved in 30.0 L water and mixed with 10.00 kg (32.61 mol) racemic cis-3-(4-methyl-piperazin-1-yl)-cyclohexylamine trihydrochloride (E), before a solution of 8.13 kg (32.61 mol) benzyloxycarbonyloxysuccinimide in 50.0 L toluene was metered in at a temperature of 25° C.±5° C. After approx. 30 minutes' stirring at 25° C.±5° C., 30.0 L water was added and the mixture was stirred for approx. 5 minutes more. After phase separation 40.0 L solvent were distilled off from the organic phase, before 60.0 L isopropyl acetate was added to the residue at 65° C.±5° C. Then at ambient temperature the solution was metered in to the mixture of 3.79 kg (16.31 mol) (1S)-(+)-camphorsulphonic acid and 0.29 L water and the mixture was refluxed until a solution was obtained. The reaction solution was cooled to 75° C.±5° C., inoculated with 10.0 g seed crystals and cooled to ambient temperature within approx. 3 hours. After the suspension had been stirred for approx. a further 3 hours at ambient temperature, the crude product was separated off and washed twice with 15.0 L isopropyl acetate. After recrystallisation from 64.0 L isopropyl acetate and 4.0 L ethanol the product (G) was dried at 50° C.±5° C.

Yield: 6.45 kg (34% of theory)

melting point: 127-129° C.

### EXAMPLE 1.4

### Methyl-[(1S,3R)-3-(4-methyl-piperazin-1-yl)-cyclohexyl]-amine trihydrochloride (I)

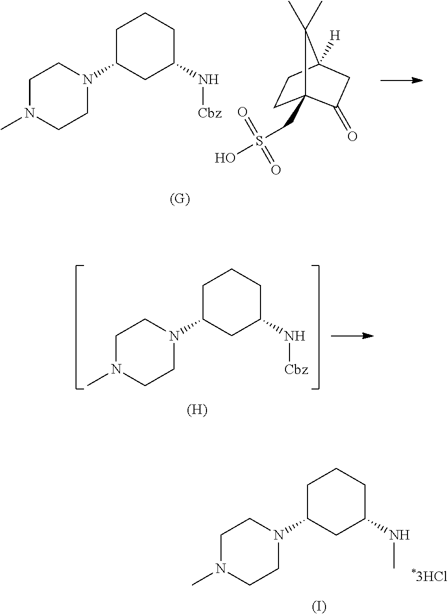

10.00 kg (17.19 mol) [(1S,3R)-3-(4-methyl-piperazin-1-yl)-cyclohexyl]carbamidic acid-benzyl ester [(1S,4R)-7,7-dimethyl-2-oxo-bicyclo[2.2.1]hept-1-yl]-methanesulphonate (G) were suspended in 50.0 L water and 50.0 L toluene and mixed with 1.44 kg (18.05 mol) sodium hydroxide solution (50%, industrial grade). After approx. 5 minutes stirring at ambient temperature the aqueous phase was separated off and 40.0 L solvent were distilled off from the organic phase in vacuo. Then the residue was combined with 10.0 L toluene and 16.0 L tetrahydrofuran and the solution was added at 85° C.±5° C. within approx. 30 minutes to the mixture of 9.46 kg (24.93 mol) lithium aluminium hydride (10% in tetrahydrofuran), 4.0 L tetrahydrofuran and 34 L toluene. After approx. 30 minutes stirring it was cooled to 35° C.±5° C., and a mixture of 0.9 L water in 2.8 L tetrahydrofuran, 0.33 kg (4.13 mol) sodium hydroxide solution (50%, industrial grade) in 0.7 L water and 2.8 L water were added successively. Then the suspension was filtered and combined with 16.0 L toluene, before 80.0 L solvent were distilled off in vacuo. 33.0 L methanol were added to the residue, it was cooled to 25° C.±5° C. and at this temperature 4.50 kg (48.13 mol, 10M) ethanolic hydrochloric acid and 16.0 L toluene were added. The product (I) was filtered off and washed twice with a 2:1 mixture of toluene and methanol and dried in vacuo.

Yield: 4.58 kg (83% of theory)

melting point: 279-282° C.

R_{f}=0.81 (CH₂Cl₂/MeOH=7/3) for (H)

### EXAMPLE 1.5

### tert-butyl[(1S,3R)-3-(4-methyl-piperazin-1-yl)-cyclohexyl]-carbamidate[(1S,4R)-7,7-dimethyl-2-oxo-bicyclo[2.2.1]hept-1-yl]-methanesulphonate (K)

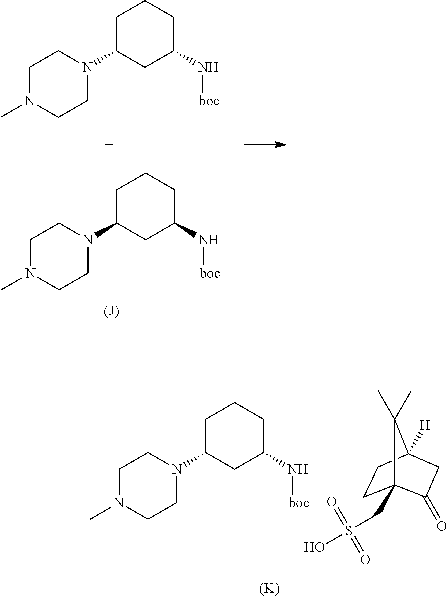

5.00 g (16.8 mmol) tert-butyl rac-cis-[3-(4-methyl-piperazin-1-yl)-cyclohexyl]carbamidate (J) were suspended in 40 mL isopropyl acetate and heated to 50° C. Then 2 mL ethanol and 2.00 g (8.61 mol) (1S)-(+)-camphorsulphonic acid were added. After it was all dissolved, the reaction solution was cooled to ambient temperature, the precipitate formed was filtered off and washed with isopropyl acetate. The colourless product (K) was dried at 50° C. in vacuo.

Yield: 2.90 g (32% of theory)

### EXAMPLE 2.1

### rac-cis-5-(4-methyl-piperazin-1-yl)-cyclohex-3-ene-carboxylic acid (M)

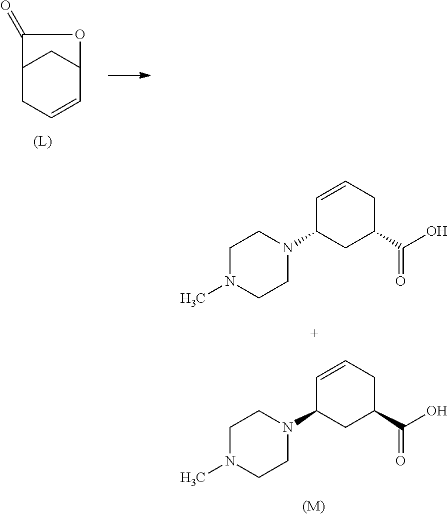

2.50 g (20.1 mmol) 6-oxa-bicyclo[3.2.1]oct-3-en-7-one (L) were dissolved in 25 mL tetrahydrofuran and cooled to 0° C.±5° C. After the addition of 223 mg (0.20 mmol) tetrakis(triphenylphosphine)palladium(0) and 10 mL water, 2.12 g (21.1 mmol) 1-methylpiperazine were slowly metered in and the reaction mixture was heated to ambient temperature overnight. Then 25 mL toluene were added and the aqueous phase was separated off. The organic phase was washed with 10 mL. The combined aqueous phases were mixed with activated charcoal, briefly stirred and filtered. The filtrate was evaporated to dryness in vacuo at 60° C.±5° C. and distilled with isopropanol. The residue was suspended in 25 mL ethyl acetate and refluxed for 30 minutes. After cooling to ambient temperature the suspension was filtered off and the product (M) was dried in vacuo.

Yield: 3.67 g (82% of theory)

melting point: 172-175° C.

R_{f}=0.36 (CH₂Cl₂/MeOH/HCOOH=7/3/0.2) for rac-cis-(M)

R_{f}=0.10 (CH₂Cl₂/MeOH/HCOOH=7/3/0.2) for rac-trans-(M)

### EXAMPLE 2.2

### rac-cis-3-(4-methyl-piperazin-1-yl)-cyclohexanecarboxylic acid (N)

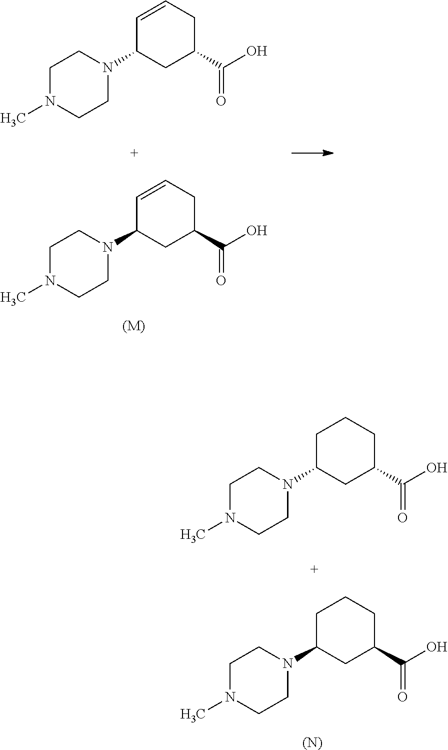

4.95 g (22.1 mmol) rac-cis-5-(4-methyl-piperazin-1-yl)-cyclohex-3-ene-carboxylic acid (M) were dissolved in 100 mL methanol and combined with 0.5 g Pd/C (10%). Then the mixture was hydrogenated at ambient temperature and at 50 PSI until all the hydrogen had been absorbed. The reaction mixture was filtered and the residue was washed with 20 mL methanol, before the solvent was eliminated completely in vacuo. The solid residue was combined with 30 mL ethyl acetate and evaporated to dryness once more. The crude product obtained (N) was suspended in 50 mL boiling ethyl acetate and after cooling to ambient temperature it was filtered off, washed with 20 mL ethyl acetate and dried in vacuo.

Yield: 4.37 g (87% of theory)

melting point: 181-184° C.

R_{f}=0.36 (CH₂Cl₂/MeOH/HCOOH=7/3/0.2) for (N)

R_{f}=0.10 (CH₂Cl₂/MeOH/HCOOH=7/3/0.2) for rac-trans-(N)

### EXAMPLE 2.3

### tert-butyl rac-cis-[3-(4-methyl-piperazin-1-yl)-cyclohexyl]-carbamidate (O)

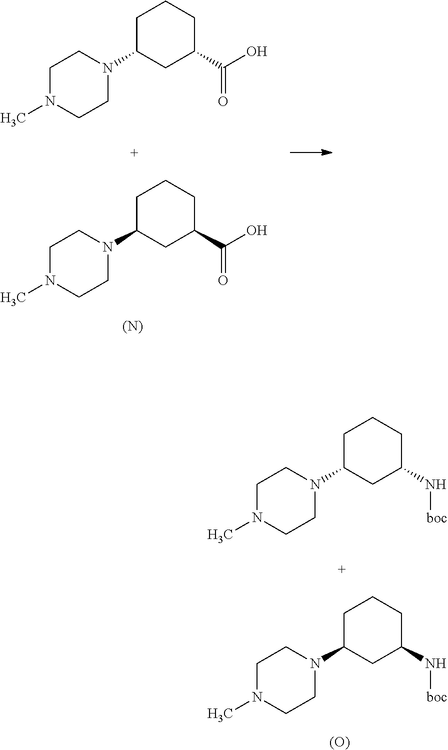

8.00 g (35.3 mmol) rac-cis-3-(4-methyl-piperazin-1-yl)-cyclohexanecarboxylic acid (N) were suspended in 120 mL toluene. Then the mixture was heated to boiling and 13 mL solvent were eliminated using the water separator. After cooling to 80° C.±5° C., 5.90 mL (42.3 mmol) triethylamine, 10.20 g (36.3 mmol) diphenylphosphorylazide (DPPA) and 10 mL toluene were added one after the other. After the reaction solution had been stirred for approx. 1 hour at 80° C.±5° C., it was transferred into a dropping funnel and slowly metered in at 25° C.±5° C. to the suspension of 8.30 g (72.5 mmol) KOtBu in 30 mL toluene. After approx. 1.5 hours stirring at ambient temperature 40 mL water were added. The aqueous phase was separated off and the organic phase was washed again with 40 mL water. Organic phases were evaporated to dryness in vacuo and the product (O) was dried in vacuo.

Yield: 9.14 g (87% of theory)

melting point: 101-104° C.

R_{f}=0.79 (CH₂Cl₂/MeOH/HCOOH=7/3/0.2) for (O)

### EXAMPLE 2.4

### tert-butyl rac-cis-[5-(4-methyl-piperazin-1-yl)-cyclohex-3-enyl]-carbamidate (Q)

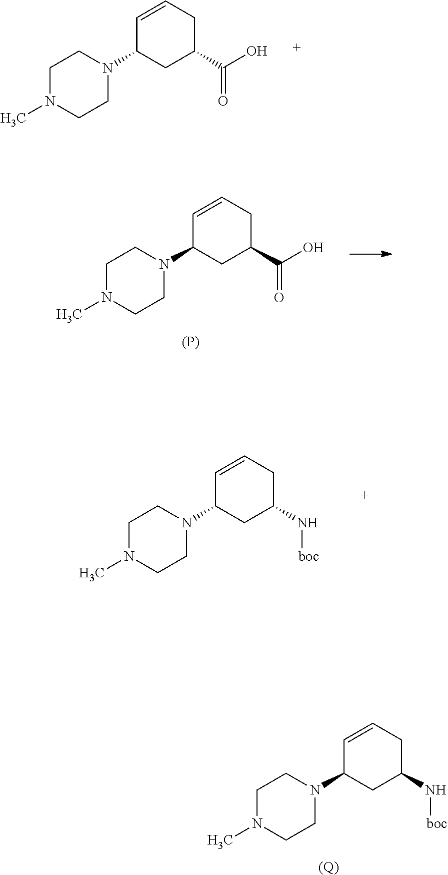

2.00 g (8.92 mmol) rac-cis-5-(4-methyl-piperazin-1-yl)-cyclohex-3-ene-carboxylic acid (P) were suspended in 50 mL toluene. Then the mixture was heated to boiling and 13 mL solvent were eliminated using the water separator. After cooling to 75° C.±5° C., 1.50 mL (10.8 mmol) triethylamine and 2.75g (9.81 mmol) diphenylphosphorylazide (DPPA) were added successively. After the reaction solution had been stirred for approx. 1.5 hours at 80° C.±5° C., it was transferred into a dropping funnel and slowly metered into the suspension of 2.19 g (19.1 mmol) KOtBu in 20 mL toluene while cooling with ice and the dropping funnel was rinsed with 10 mL toluene. After approx. 1.5 hours' stirring at ambient temperature, 40 mL water were added. The aqueous phase was separated off and the organic phase was washed again with 20 mL water. Finally the organic phase was to evaporated to dryness in vacuo and yielded product (Q).

Yield: 2.25 g (85% of theory)

R_{f}=0.84 (CH₂Cl₂/MeOH=7/3)

### EXAMPLE 2.5

### Benzyl rac-cis-[3-(4-methyl-piperazin-1-yl)-cyclohexyl]-carbamidate (S)

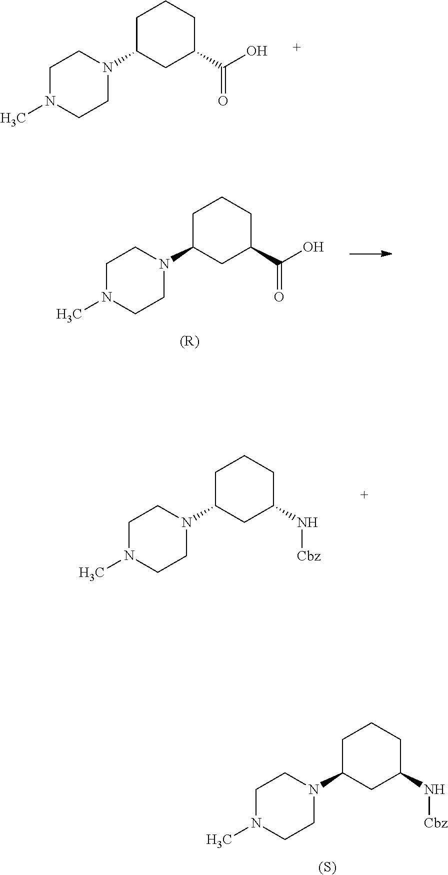

6.00 g (26.5 mmol) rac-cis-3-(4-methyl-piperazin-1-yl)-cyclohexanecarboxylic acid (R) were suspended in 120 mL toluene. Then the mixture was heated to boiling and 13 mL solvent were eliminated using the water separator. After cooling to 80±5° C., 4.40 mL (42.3 mmol) triethylamine, 10.20 g (31.5 mmol) diphenylphosphorylazide (DPPA) and 10 mL toluene were added in succession. After the reaction solution had been stirred for approx. 1 hour at 80±5° C., 3.50 mL (32.3 mmol) benzylalcohol and 10 mL toluene were metered in. After cooling to 40±5° C., 60 mL water and 60 mL ethyl acetate were added. The organic phase was separated off and washed with 60 mL water. After drying through Na₂SO₄ the solvent was eliminated in vacuo.

Yield: 7.85 g (89% of theory)

1. A method for preparing a compound of the formula I 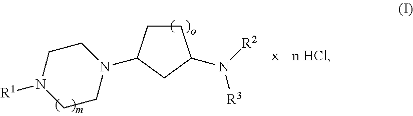
wherein
m denotes one of the numbers 1 or 2,
n denotes one of the numbers 0, 1, 2 or 3,
o denotes one of the numbers 0, 1, 2 or 3,
R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
R² denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms, and
R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)— or
(e) C₁₋₄-alkyl-C(O), which may be substituted by 1, 2 or 3 fluorine or chlorine atoms,
or an enantiomer or diastereomer thereof, comprising the following steps:
(a) addition of a compound of the formula III 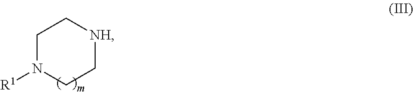
wherein m and R¹ are defined as mentioned hereinbefore, to a compound of the formula IV 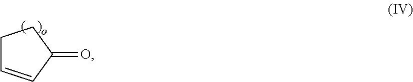
wherein o is defined as mentioned hereinbefore;
(b) reaction of a compound of the formula V obtained in step (a) 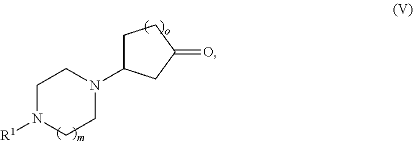
wherein m, o and R¹ are defined as mentioned hereinbefore, with hydroxylamine-hydrochloride;
(c) reduction of an oxime of the formula VI obtained in step (b) 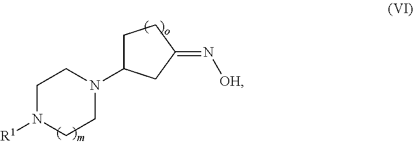
wherein m, o and R¹ are defined as mentioned hereinbefore, in the presence of a catalyst;
(d) optionally isolation of a compound of the formula Ia obtained in step (c) 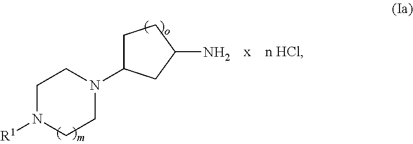
wherein m, n, o and R¹ are defined as mentioned hereinbefore;
(e) coupling an amine of the formula Ia obtained in step (c) or (d) 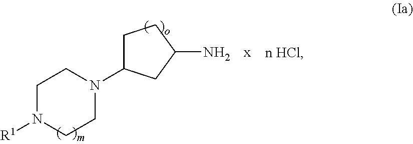
wherein m, n, o and R¹ are defined as mentioned hereinbefore, to a compound of the formula VII
X—R², (VII)
wherein R² is defined as mentioned hereinbefore and X denotes a leaving group;
(f) optionally isolating a compound of the formula Ib obtained in step (e) 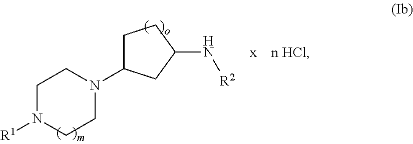
wherein m, n, o, R¹ and R² are defined as mentioned hereinbefore;
(g) optionally again coupling a compound of the formula Ib obtained in step (e) or (f) 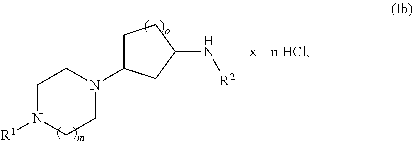
wherein m, n, o, R¹ and R² are defined as mentioned hereinbefore, to a compound of the formula VIII
X—R³, (VIII)
wherein R³ is defined as mentioned hereinbefore and X denotes a leaving group;
(h) optionally isolating a compound of the formula I obtained in step (g);
(i) optionally stereoselectively separating or concentrating the stereoisomers of a compound of the formula Ia obtained in step (c) or (d) or of a compound of the formula Ib obtained in step (e) or (f) or of a compound of the formula I obtained in step (g) or (h), by co-crystallisation or salt formation with inorganic acids or chiral acids;
(j) optionally isolating one or more stereoisomers of the formula IX obtained in step (i) 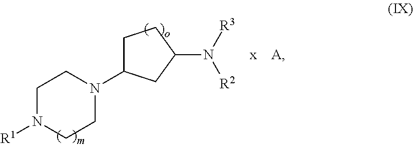
wherein m, n, o, R¹, R² and R³ are defined as mentioned hereinbefore and A denotes one or more chiral acids or one or more corresponding anions of one or more inorganic acids;
(k) reacting a compound of the formula IX obtained in step (i) or (j) with a base;
(l) optionally isolating a stereoisomeric or enantiomerically enriched compound of the formula I 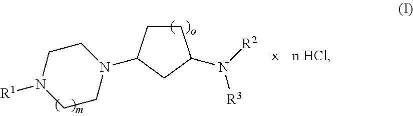
wherein m, n, o, R¹, R² and R³ are defined as mentioned hereinbefore; and
(m) optionally subsequently eliminating an amine protecting group in a compound of the formula I thus obtained wherein m, n and o are defined as mentioned hereinbefore and at least one of the groups R¹, R² or R³ carries an amine protecting group, thus obtaining a compound of the formula I wherein m, n and o are defined as mentioned hereinbefore and at least one of the groups R¹, R² or R³ denotes a hydrogen atom; and
(n) optionally reducing a compound of the formula I thus obtained wherein m, n, o and R¹ are defined as mentioned hereinbefore and at least one of the groups R² or R³ denotes a C₁₋₄-alkyl-O—C(O)— or benzyl-O—C(O)— group, with a reducing agent, thus obtaining a compound of the formula I wherein m, n, o and R¹ are defined as mentioned hereinbefore and at least one of the groups R² or R³ denotes a methyl group.

2. The method according to claim 1, wherein,
m denotes one of the numbers 1 or 2,
n denotes one of the numbers 0, 1, 2 or 3,
o denotes one of the numbers 0, 1 or 2,
R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
R² denotes
(a) H,
(b) C₁₋₄-alkyl, C₃₋₆-cycloalkyl,
(c) benzyl,
(d) C₁₋₄-alkyl-O—C(O)— or benzyl-O—C(O)— and
R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—.

3. The method of claim 1 wherein,
m denotes one of the numbers 1 or 2,
n denotes one of the numbers 0, 1 or 2,
o denotes one of the numbers 0, 1 or 2,
R¹ denotes H, C₁₋₄-alkyl, C₃₋₆-cycloalkyl, C₁₋₄-alkyl-O—C(O)—, benzyl-O—C(O)— or benzyl, and
R² denotes
(a) H,
(b) benzyl,
(c) C₁₋₄-alkyl-O—C(O)— or benzyl-O—C(O)—, and
R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—.

4. The method of claim 1, wherein,
m denotes one of the numbers 1 or 2,
n denotes one of the numbers 1 or 2,
o denotes one of the numbers 0, 1 or 2,
R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and
R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—.

5. The method of claim 1, wherein,
m denotes one of the numbers 1 or 2,
n denotes the number 0,
o denotes one of the numbers 0, 1 or 2,
R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and
R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—.

6. The method of claim 1, wherein,
m denotes one of the numbers 1 or 2,
n denotes the number 3,
o denotes one of the numbers 0, 1 or 2,
R¹ denotes
(a) H,
(b) C₁₋₄-alkyl, C₃₋₆-cycloalkyl,
(c) benzyl,
(d) C₁₋₄-alkyl-O—C(O)— or benzyl-O—C(O)—,
R² denotes
(a) H,
(b) C₁₋₄-alkyl, C₃₋₆-cycloalkyl,
(c) benzyl,
(d) C₁₋₄-alkyl-O—C(O)— or benzyl-O—C(O)—, and
R³ denotes
(a) H,
(b) C₁₋₄-alkyl, C₃₋₆-cycloalkyl,
(c) benzyl,
(d) C₁₋₄-alkyl-O—C(O)— or benzyl-O—C(O)—.

7. The method of claim 1, wherein,
R² denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl and
R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trifluoroacetyl or trichloroacetyl.

8. The method of claim 1, wherein,
m denotes the number 1,
n denotes one of the numbers 0, 1, 2 or 3,
o denotes one of the numbers 1 or 2,
R¹ denotes H, CH₃, benzyl, tert.butyl-O—C(O)— or benzyl-O—C(O)—,
R² denotes H, CH₃, benzyl, tert.butyl-O—C(O)— or benzyl-O—C(O)—,
R³ denotes H, CH₃, benzyl, tert.butyl-O—C(O)— or benzyl-O—C(O)—.

9. A method for preparing a compound of the formula I 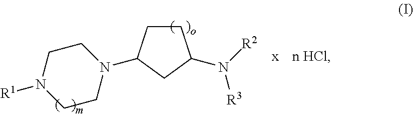
wherein
m denotes one of the numbers 1 or 2,
n denotes one of the numbers 0, 1, 2 or 3,
o denotes the number 2,
R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
R² denotes
(a) H,
(b) C₁₋₄-alkyl-O—C(O)—, benzyl-O—C(O)— and
R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—
(e) acetyl, trifluoroacetyl or trichloroacetyl,
or an enantiomer or diastereomer thereof, comprising the following steps:
(a1) reacting the compound of the formula 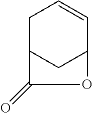
with a compound of the formula III 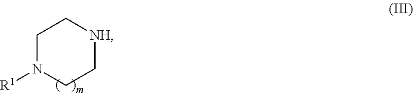
wherein m and R¹ are defined as mentioned hereinbefore, in the presence of a catalyst;
(a2) reducing a compound of the formula X obtained in step (a1) 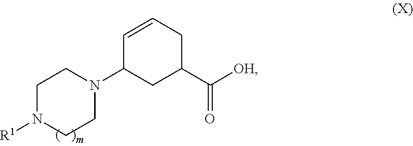
wherein m and R¹ are defined as mentioned hereinbefore;
(a3) reacting a compound of the formula XI obtained in step (a2) 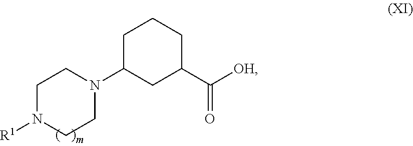
wherein m and R¹ are defined as mentioned hereinbefore, in the presence of a base, with an azide source, and catching the resulting intermediate compound of the formula XII 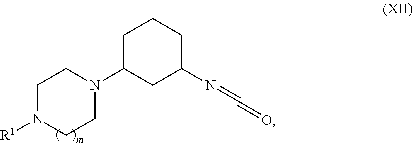
wherein m and R¹ are defined as mentioned hereinbefore, with a compound of the formula XIII
HO—R⁴, (XIII)
wherein R⁴ denotes a hydrogen atom, a C₁₋₄-alkyl or benzyl group; and
(a4) optionally isolating a compound of the formula Ib obtained in step (a3) 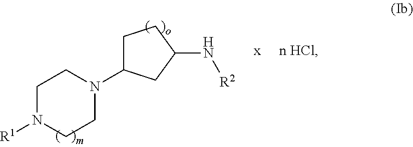
wherein m, n, o, R¹ and R² are defined as mentioned hereinbefore.

10. The method of claim 9, wherein,
m denotes the number 1,
n denotes one of the numbers 0, 1, 2 or 3,
o denotes the number 2,
R¹ denotes H, CH₃, benzyl, tert.butyl-O—C(O)— or benzyl-O—C(O)—,
R² denotes H, tert.butyl-O—C(O)— or benzyl-O—C(O)—,
R³ denotes H, CH₃, benzyl, tert.butyl-O—C(O)— or benzyl-O—C(O)—.

11. A method for preparing a compound of the formula I 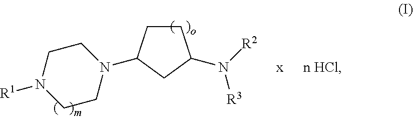
wherein
m denotes one of the numbers 1 or 2,
n denotes one of the numbers 0, 1, 2 or 3,
o denotes the number 2,
R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
R² denotes
(a) H,
(b) C₁₋₄-alkyl-O—C(O)—, benzyl-O—C(O)— and
R³ denotes
(a) H,
(b) C₁₋₄-alkyl, C₃₋₆-cycloalkyl,
(c) benzyl,
(d) C₁₋₄-alkyl-O—C(O)—, benzyl-O—C(O)—
(e) acetyl, trifluoroacetyl or trichloroacetyl,
or an enantiomer of diastereomer thereof, comprising the following steps:
(b1) reacting the compound of the formula 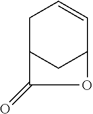
with a compound of the formula III 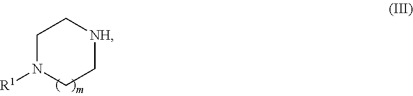
wherein m and R¹ are defined as mentioned hereinbefore, in the presence of a catalyst;
(b2) reacting a compound of the formula X obtained in step (b1) 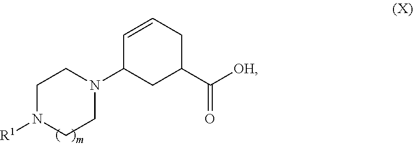
wherein m and R¹ are defined as mentioned hereinbefore, in the presence of a base with an azide source, and catching the resulting intermediate compound of the formula XIV 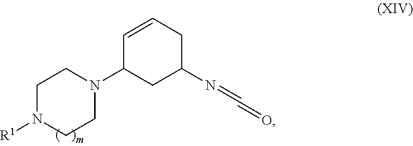
wherein m and R¹ are defined as mentioned hereinbefore, with a compound of the formula XIII
HO—R⁴, (XIII)
wherein R⁴ denotes a hydrogen atom, a C₁₋₄-alkyl or benzyl group;
(b3) optionally isolating a compound of the formula XV obtained in step (b2) 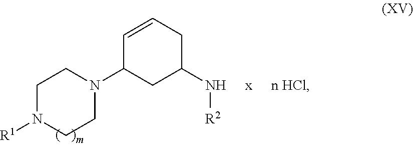
wherein m, n, R¹ and R² are defined as mentioned hereinbefore;
(b4) reducing a compound of the formula XV obtained in step (b2) or (b3) 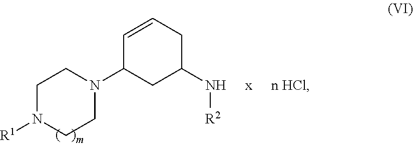
wherein m and R¹ are defined as mentioned hereinbefore; and
(b5) optionally isolating a compound of the formula I obtained in step (b4) 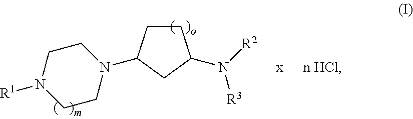
wherein m, n, R¹ and R² are defined as mentioned hereinbefore.

12. The method according to claim 11, wherein,
m denotes the number 1,
n denotes one of the numbers 0, 1, 2 or 3,
o denotes the number 2,
R¹ denotes H, CH₃, benzyl, tert.butyl-O—C(O)— or benzyl-O—C(O)—,
R² denotes H, tert.butyl-O—C(O)— or benzyl-O—C(O)—,
R³ denotes H, CH₃, benzyl, tert.butyl-O—C(O)— or benzyl-O—C(O)—.

13. A compound of the formula X 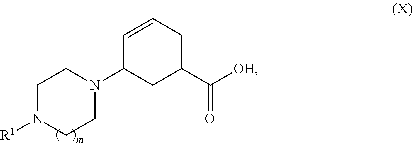
wherein
m denotes one of the numbers 1 or 2 and
R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trichloroacetyl or trifluoroacetyl,
or an enantiomer, diastereomer or a salt thereof.

14. A compound of the formula X according to claim 13, selected from the group consisting of:

| No. | Structure |
|---|---|
| (1) | 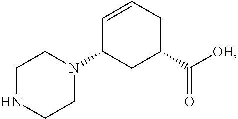 |
| (2) | 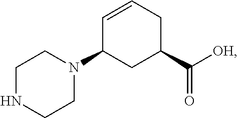 |
| (3) | 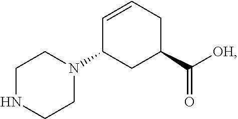 |
| (4) | 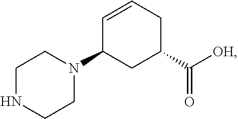 |
| (5) | 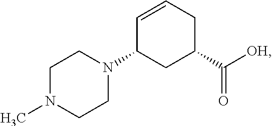 |
| (6) | 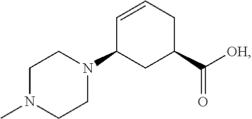 |
| (7) | 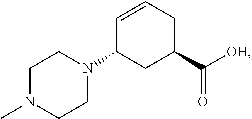 |
| (8) | 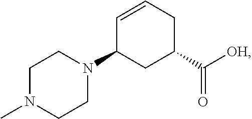 |
| (9) | 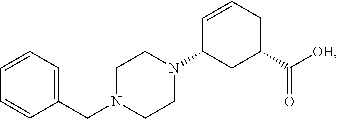 |
| (10) | 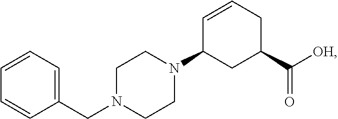 |
| (11) | 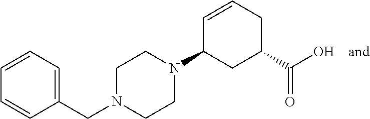 |
| (12) | 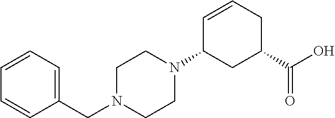 |

or a salt, enantiomer or diastereomer thereof.

15. A compound of the formula XI 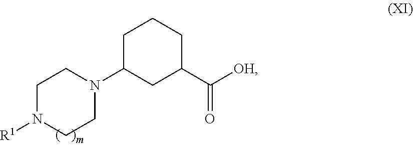
wherein
m denotes one of the numbers 1 or 2 and
R¹ denotes
(a) H,
(b) C₁₋₄-alkyl, C₃₋₆-cycloalkyl,
(c) benzyl,
(d) C₁₋₄-alkyl-O—C(O)—, benzyl-O—C(O)—,
(e) acetyl, trichloroacetyl or trifluoroacetyl,
or a salt, enantiomer or diastereomer thereof.

16. A compound of the formula XI according to claim 15, selected from the group consisting of:

| No. | Structure |
|---|---|
| (1) | 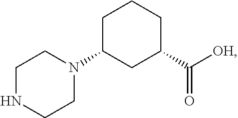 |
| (2) | 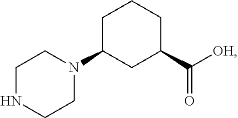 |
| (3) | 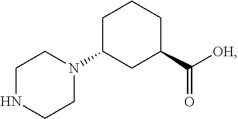 |
| (4) | 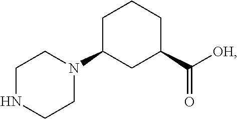 |
| (5) | 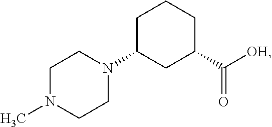 |
| (6) | 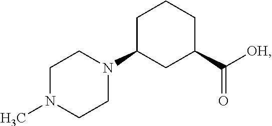 |
| (7) | 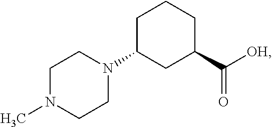 |
| (8) | 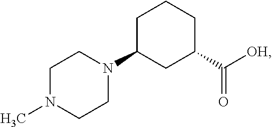 |
| (9) | 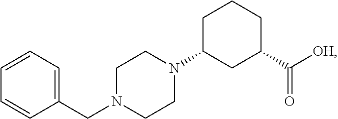 |
| (10) | 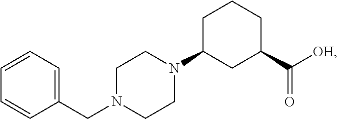 |
| (11) | 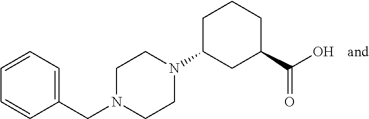 |
| (12) | 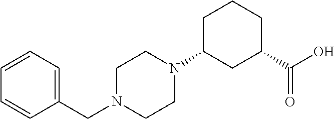 |

or an enantiomer, diastereomer or salt thereof.
